(19) United States
(12) Patent Application Publication
(10) Pub. No.: US 2024/0226165 A1
(43) Pub. Date: Jul. 11, 2024

(54) Methods of Producing Improved Immune Cell Populations

(71) Applicant: Prescient Therapeutics Ltd, South Melbourne, Victoria (AU)

(72) Inventors: Steven L. Yatomi-Clarke, South Melbourne (AU); Rebecca Lim, South Melbourne (AU); Said M. Sebti, South Melbourne (AU); Phillip K. Darcy, Melbourne (AU); Jasmine Li, Melbourne (AU)

(21) Appl. No.: 18/562,252

(22) PCT Filed: May 18, 2022
(86) PCT No.: PCT/AU2022/050479

§ 371 Date: Nov. 17, 2023

(30) Foreign Application Priority Data
May 19, 2021 (AU) 2021901496

(51) Int. Cl. A61K 35/17; A61K 9/00; A61K 31/7064; A61K 39/00; A61P 35/00; C07K 16/28; C07K 16/32; C12N 5/0783; C12N 5/0786; C12N 15/85

(52) U.S. Cl. CPC A61K 35/17; A61K 9/0019; A61K 31/7064; A61K 39/4611; A61K 39/4631; A61K 39/464406; A61K 39/464838; A61P 35/00; C07K 16/2818; C07K 16/32; C12N 5/0636; C12N 5/0645; C12N 5/0646; C12N 15/85; A61K 2039/505; A61K 2239/13; A61K 2239/48; C12N 2501/999

(57) ABSTRACT

The present invention relates to methods of producing improved immune cell populations.

The present application claims priority from AU2021901496, filed 19 May 2021, the entire contents of which is incorporated herein by reference.

### FIELD OF THE INVENTION

The present invention relates to methods of producing improved immune cell populations.

### BACKGROUND OF THE INVENTION

Chimeric antigen receptor (CAR) T-cell therapy has made remarkable strides in the treatment of patients with difficult to treat cancers, however, poor CAR-T persistence remains a critical challenge. Poor persistence of infused CAR-T-cells is inversely correlated with durable clinical remissions in patients with cancers. The frequency of CAR-T-cells with naïve, central memory (T_{CM}) or stem cell (T_{SCM}) phenotype have been shown to be an important predictive factor of clinical efficacy, with the ability to achieve better persistence CAR-T-cells with effector (T_{E}) and effector memory (TEM) phenotypes (McLellan and Ali Hosseini Rad, 2019). While TE and TEM cells display superior tumour killing capacity in vitro, they have low self-renewal capacity, diminished ability for niche homing and survival, and are more vulnerable to activation induced cell death (AICD) or exhaustion (McLellan and Ali Hosseini Rad, 2019).

Thus, there is a need for improved populations of immune cells, such as CAR-T-cell populations for use in CAR-T therapy.

### SUMMARY OF THE INVENTION

The present inventors have shown that AKT inhibitors and/or inhibitors of a PH domain protein, when administered in vivo, can improve the efficacy of conventional CAR-T therapies in subjects requiring treatment thereof. The present inventors have also shown that AKT inhibitors and/or inhibitors of a PH domain protein can be used to improve properties of cultured immune cells.

Thus, in one aspect, the present invention provides a method of modifying an immune response in a subject, the method comprising administering to the subject a population of immune cells, wherein the immune cells were produced using a method comprising culturing immune cells in medium comprising an AKT inhibitor and/or an inhibitor of a PH domain protein, preferably wherein the immune cells are T-cells, dendritic cells, natural killer cells, myeloid cells, macrophages or a combination thereof.

In an embodiment, the method is for modifying a T-cell immune response, dendritic cell immune response, natural killer cell immune response or a myeloid cell or macrophage immune response in a subject.

In a further aspect, the present invention provides use of a population of immune cells in the preparation of a medicament for modifying an immune response in a subject, wherein the immune cells were produced using a method comprising culturing immune cells in medium comprising an AKT inhibitor and/or an inhibitor of a PH domain protein, preferably wherein the immune cells are T-cells, dendritic cells, natural killer cells, myeloid cells, macrophages or a combination thereof.

In a further aspect, the present invention provides a population of immune cells for use in modifying an immune response in a subject, wherein the immune cells were produced using a method comprising culturing immune cells in medium comprising an AKT inhibitor and/or an inhibitor of a PH domain protein, preferably wherein the immune cells are T-cells, dendritic cells, natural killer cells, myeloid cells, macrophages or a combination thereof.

In a further aspect, the present invention provides a method of modifying a T-cell response in a subject, the method comprising administering to the subject a population of T-cells comprising a chimeric antigen receptor (CAR-T-cells), wherein the CAR-T-cells were produced using a method comprising culturing CAR-T-cells in medium comprising an AKT inhibitor and/or an inhibitor of a PH domain protein.

In a further aspect, the present invention provides use of a population of T-cells comprising a chimeric antigen receptor (CAR-T-cells) in the preparation of a medicament for modifying a T-cell response in a subject, wherein the CAR-T-cells were produced using a method comprising culturing CAR-T-cells in medium comprising an AKT inhibitor and/or an inhibitor of a PH domain protein.

In a further aspect, the present invention provides a population of T-cells comprising a chimeric antigen receptor (CAR-T-cells) for use in modifying a T-cell response in a subject, wherein the CAR-T-cells were produced using a method comprising culturing CAR-T-cells in medium comprising an AKT inhibitor and/or an inhibitor of a PH domain protein.

In a further aspect, the present invention provides a method of modifying an immune response in a subject, preferably a T-cell response, the method comprising:
a) administering to the subject an AKT inhibitor and/or an inhibitor of a PH domain protein, and
b) at least about 18 hours after step a) administering to the subject a population of immune cells, preferably comprising T-cells comprising a chimeric antigen receptor (CAR-T-cells).

In a further aspect, the present invention provides use of an AKT inhibitor and/or an inhibitor of a PH domain protein in the preparation of a medicament for modifying an immune response in a subject, preferably a T-cell response, the treatment comprising:
a) administering to the subject an AKT inhibitor and/or an inhibitor of a PH domain protein, and
b) at least about 18 hours after step a) administering to the subject a population of immune cells, preferably comprising T-cells comprising a chimeric antigen receptor (CAR-T-cells).

In a further aspect, the present invention provides an AKT inhibitor and/or an inhibitor of a PH domain protein for use in modifying an immune response in a subject, preferably a T-cell response, the treatment comprising:
a) administering to the subject an AKT inhibitor and/or an inhibitor of a PH domain protein, and
b) at least about 18 hours after step a) administering to the subject a population of immune cells, preferably comprising T-cells comprising a chimeric antigen receptor (CAR-T-cells).

In an embodiment, the modification of an immune response or modification of a T-cell response comprises the enrichment of central memory cells (T_{CM}). In an embodiment, the T_{CM} cells include CD45RO+ CD62L+ T-cells, preferably CD45RO+ CD62L^{hi} T-cells.

In a further aspect, the present invention provides a method of modifying a dendritic cell and/or natural killer cell response in a subject, the method comprising:
a) administering to the subject an AKT inhibitor and/or an inhibitor of a PH domain protein, and
b) administering to the subject a population of cells comprising dendritic cell and/or natural killer cells.

In an embodiment, the population of cells are administered between about 18 hours and about 72 hours after the AKT inhibitor and/or an inhibitor of a PH domain protein. In an embodiment, the population of cells are administered between about 24 hours and about 72 hours after the AKT inhibitor and/or an inhibitor of a PH domain protein. In an embodiment, the population of cells are administered between about 24 hours and about 48 hours after the AKT inhibitor and/or an inhibitor of a PH domain protein.

The present inventors have determined that AKT inhibitors and/or an inhibitors of a PH domain protein reduce the killing activity of CAR-T-cells, and hence can be used to reduce the occurrence of cytokine release syndrome (CRS). Thus, in a further aspect, the present invention provides a method of reducing cytokine release syndrome (CRS) in a subject undergoing CAR-T-cell therapy, the method comprising administering to the subject an AKT inhibitor and/or an inhibitor of a PH domain protein and/or the CAR-T-cells, wherein the CAR-T-cells have been cultured in a medium comprising the AKT inhibitor and/or an inhibitor of a PH domain protein.

In a further aspect, the present invention provides use of an AKT inhibitor and/or an inhibitor of a PH domain protein and/or CAR-T-cells in the preparation of a medicament for reducing cytokine release syndrome (CRS) in a subject undergoing CAR-T-cell therapy, wherein the CAR-T-cells have been cultured in a medium comprising the AKT inhibitor and/or an inhibitor of a PH domain protein.

In a further aspect, the present invention provides an AKT inhibitor and/or an inhibitor of a PH domain protein and/or CAR-T-cells for use in reducing cytokine release syndrome (CRS) in a subject undergoing CAR-T-cell therapy, wherein the CAR-T-cells have been cultured in a medium comprising the AKT inhibitor and/or an inhibitor of a PH domain protein.

In an embodiment, the chimeric antigen receptor comprises a CD28z co-stimulatory domain.

Examples of AKT inhibitors and/or inhibitors of a PH domain protein which can be used for the invention include, but are not limited to, one or more selected from: triciribine (TCN), triciribine 5′-monophosphate (TCN-P), AKT inhibitor VIII, MK-2206, AZD5363, GDC-0068, GSK2141795 and GSK2110183 hydrochloride.

In an embodiment, the AKT inhibitor and/or an inhibitor of a PH domain protein is TCN or TCN-P.

In an embodiment, the immune cells or CAR-T cells are administered at a dosage of about 0.2 million per kg, about 0.5 million per kg, about 0.7 million per kg, about 1.0 million per kg, about 1.2 million per kg, about 1.5 million per kg, about 1.7 million per kg, about 2.0 million per kg, about 2.2 million per kg, about 2.5 million per kg, about 2.7 million per kg, about 3.0 million per kg or higher. In another embodiment, the immune cells or CAR-T cells are administered at a dosage of between about 0.2-0.5 million per kg, between about 0.5-0.7 million per kg, between about 0.7-1.0 million per kg, between about 1.0-1.2 million per kg, between about 1.2-1.5 million per kg, between about 1.5-1.7 million per kg, between about 1.7-2.0 million per kg, between about 2.0-2.2 million per kg, between about 2.2-2.5 million per kg, between about 2.5-2.7 million per kg or between about 2.7-3.0 million per kg. In another embodiment, the immune cells or CAR-T cells are administered at a dosage of 0.5-2 million per kg.

In an embodiment, the subject is immunodepleted. Examples of methods to provide immunodepletion include, but are not limited to, lymphodepleting chemotherapy or radiation therapy.

In an embodiment, the subject has a cancer, an infection, or an inflammatory disease.

In an embodiment, the infection is a bacterial, fungal, protozoan or viral infection. In an embodiment, the infection is a viral infection. In an embodiment, the viral infection is a chronic viral infection such as an infection with a Hepatitis C virus (HCV), hepatitis B (HCB), human papilloma virus (HPV), cytomegalovirus (CMV), Epstein-Barr virus (EBV), varicella zoster virus, coxsackie virus, or human immunodeficiency virus (HIV).

In an embodiment, the subject has a cancer. In an embodiment, the subject has a solid tumour, such as breast or colon cancer.

In an embodiment, the subject has a cancer associated with low antigen abundance. In an embodiment, the subject has
i) acute myeloid leukaemia where low CD33+ blasts are dominant, or
ii) diffuse large B cell lymphoma or non-Hodgkin's lymphoma with low levels of CD19 and/or CD20.

In an embodiment, the subject is an animal. In an embodiment, the subject is a mammal. In an embodiment, the subject is a human.

Also provided is the use of an AKT inhibitor and/or an inhibitor of a PH domain protein for the manufacture of a medicament for modifying an immune response in a subject, preferably a T-cell response, wherein the subject will be administered with a population of immune cells, preferably comprising T-cells comprising a chimeric antigen receptor (CAR-T-cells) at least 18 hours after the medicament.

Also provided is the use of a population of immune cells, preferably comprising T-cells comprising a chimeric antigen receptor (CAR-T-cells) for the manufacture of a medicament for modifying an immune response in a subject, preferably a T-cell response, wherein the subject will be or has been administered with an AKT inhibitor and/or an inhibitor of a PH domain protein at least 18 hours before the medicament.

Also provided is the use of an AKT inhibitor and/or an inhibitor of a PH domain protein for the manufacture of a medicament for modifying an immune response in a subject, preferably a T-cell response, wherein the medicament is CAR-T-cells.

Also provided is the use of an AKT inhibitor and/or an inhibitor of a PH domain protein for the manufacture of a medicament for modifying a dendritic cell and/or natural killer cell response in a subject.

Also provided is the use of a population of immune cells comprising dendritic cells and/or natural killer cells for the manufacture of a medicament for modifying a dendritic cell and/or natural killer cell response in a subject, wherein the subject has been, or will be, administered with an AKT inhibitor and/or an inhibitor of a PH domain protein.

Also provided is an AKT inhibitor and/or an inhibitor of a PH domain protein for use in producing a population of immune cells, preferably comprising T-cells comprising a chimeric antigen receptor (CAR-T-cells), for modifying an immune response in a subject, preferably a T-cell response.

Also provided is an AKT inhibitor and/or an inhibitor of a PH domain protein for use in modifying an immune response in a subject, preferably a T-cell response in a subject, wherein the subject will be administered with a population of immune cells, preferably comprising T-cells comprising a chimeric antigen receptor (CAR-T-cells) at least 18 hours after the medicament.

In an embodiment, the methods described herein further comprise the administration of a checkpoint inhibitor, preferably an anti-PD-1 antibody. Advantageously, the administration of a checkpoint inhibitor in combination with a CAR-T-cell of the invention, preferably a CAR-T-cell that has been pre-treated with an AKT inhibitor and/or an inhibitor of a PH domain protein, demonstrates a synergistic effect on tumour growth and/or survival of a subject having or suspected of having cancer.

Thus, in one aspect, there is provided a method for modifying an immune response in a subject, preferably a T-cell response in a subject, the method comprising administering to the subject a population of immune cells, preferably T-cells comprising a chimeric antigen receptor (CAR-T-cells) and a checkpoint inhibitor, preferably an anti-PD-1 antibody, preferably wherein the immune cells were produced using a method comprising culturing immune cells in medium comprising an AKT inhibitor and/or an inhibitor of a PH domain protein. Preferably, the immune cells are T-cells, dendritic cells, natural killer cells, myeloid cells, macrophages or a combination thereof. In an embodiment, the method comprises administering CAR-T-cells that have been pretreated with an AKT inhibitor and/or an inhibitor of a PH domain protein and a checkpoint inhibitor, optionally concurrently or sequentially. In this embodiment, the effect of the treatment (i.e., tumour growth and/or survival) is synergistic when compared to the individual effects of each treatment.

In another embodiment, the method comprises administering CAR-T-cells, an AKT inhibitor and/or an inhibitor of a PH domain protein and a checkpoint inhibitor, optionally concurrently or sequentially. In this embodiment, the effect of the treatment (on tumour growth and/or survival) is synergistic when compared to the individual effects of each treatment alone. In an embodiment, the CAR-T-cells are not pretreated with an AKT inhibitor and/or an inhibitor of a PH domain protein.

In another aspect, there is provided a method for modifying an immune response in a subject, preferably a T-cell response in a subject, the method comprising administering to the subject a checkpoint inhibitor, preferably an anti-PD-1 antibody and an AKT inhibitor and/or an inhibitor of a PH domain protein. In this embodiment, the effect of the treatment (on tumour growth and/or survival) is synergistic when compared to the individual effects of each treatment alone.

In another aspect, there is provided use of a population of immune cells, preferably T-cells comprising a chimeric antigen receptor (CAR-T-cells) and a checkpoint inhibitor, preferably an anti-PD-1 antibody, in the preparation of a medicament for modifying an immune response in a subject, preferably a T-cell response in a subject, wherein the immune cells were produced using a method comprising culturing immune cells in medium comprising an AKT inhibitor and/or an inhibitor of a PH domain protein.

In another aspect, there is provided use of a population of immune cells, preferably T-cells comprising a chimeric antigen receptor (CAR-T-cells) in the preparation of a medicament for modifying an immune response in a subject, preferably a T-cell response in a subject, wherein the subject has been, or will be, administered with a checkpoint inhibitor, preferably an anti-PD-1 antibody, wherein the immune cells were produced using a method comprising culturing immune cells in medium comprising an AKT inhibitor and/or an inhibitor of a PH domain protein.

In another aspect, there is provided use of a checkpoint inhibitor, preferably an anti-PD-1 antibody in the preparation of a medicament for modifying an immune response in a subject, preferably a T-cell response in a subject, wherein the subject has been, or will be, administered with of a population of immune cells, preferably T-cells comprising a chimeric antigen receptor (CAR-T-cells), wherein the immune cells were produced using a method comprising culturing immune cells in medium comprising an AKT inhibitor and/or an inhibitor of a PH domain protein.

In another aspect, there is provided a population of immune cells, preferably T-cells comprising a chimeric antigen receptor (CAR-T-cells) and a checkpoint inhibitor, preferably an anti-PD-1 antibody for use in modifying an immune response in a subject, preferably a T-cell response in a subject, wherein the immune cells were produced using a method comprising culturing immune cells in medium comprising an AKT inhibitor and/or an inhibitor of a PH domain protein.

In an embodiment, modification of the immune response or T-cell immune response increases survival of the subject when compared to a subject not receiving CAR-T-cells and/or an AKT inhibitor and/or an inhibitor of a PH domain protein. In an embodiment, survival is increased by 3, 6, 9, 12, 24, 36, 48, 60, 72, 84, 96 months or more when compared to a subject not receiving CAR-T-cells and/or AKT inhibitor and/or an inhibitor of a PH domain protein of the invention.

In an embodiment, the subject has been diagnosed as having, or is suspected of having a disease or disorder such as cancer, infection or an inflammatory disease. In an embodiment, the subject has been diagnosed as having, or is suspected of having colon cancer or breast cancer. Thus, in an embodiment, the methods described herein comprise a step of diagnosing the subject as having or suspected of having a disease or disorder such as cancer, preferably colon cancer or breast cancer, infection or an inflammatory disease.

In an embodiment, the methods or uses may further comprise the administration of an additional therapeutic agent, optionally selected from the group consisting of chemotherapy, radiotherapy, surgery, bone marrow transplant, drug therapy, cryoablation or radiofrequency ablation.

In an embodiment, the immune cells, CAR-T-cells, AKT inhibitor and/or an inhibitor of a PH domain protein and/or checkpoint inhibitor may be administered sequentially or concurrently.

The present inventors have also advantageously found that the efficacy of treatment for cancer may be increased by using an AKT inhibitor as an adjuvant in addition to during the manufacturing process of the CAR-T-cell.

Thus in an aspect, there is provided a method for modifying an immune response in a subject, preferably a T-cell immune response, the method comprising administering to the subject:
(i) a population of immune cells, preferably T-cells comprising a chimeric antigen receptor (CAR-T-cells); and
(ii) an AKT inhibitor and/or an inhibitor of a PH domain protein, wherein the immune cells were produced using a method comprising culturing immune cells in medium comprising an AKT inhibitor and/or an inhibitor of a PH domain protein.

In an embodiment, the method further comprises a step of producing a cell population comprising immune cells of the invention.

In an embodiment, the dose of AKT inhibitor and/or an inhibitor of a PH domain protein administered to the subject is about 0.5 mg/kg, about 1 mg/kg, about 1.5 mg/kg, about 2 mg/kg, about 2.5 mg/kg, about 3.0 mg/kg or higher. In another embodiment, the dose of AKT inhibitor and/or an inhibitor of a PH domain protein administered to the subject is between about 0.5 mg/kg-1.0 mg/kg, about 1.0 mg/kg-1.5 mg/kg, about 1.5 mg/kg-2.0 mg/kg, about 2.0 mg/kg-2.5 mg/kg, about 2.5 mg/kg-3.0 mg/kg or higher. Preferably, the dose of AKT inhibitor and/or an inhibitor of a PH domain protein administered to the subject is about 2 mg/kg.

In an embodiment, the AKT inhibitor and/or an inhibitor of a PH domain protein is administered intravenously to the subject once weekly, twice weekly, three times weekly, four times weekly or more. In another embodiment, the AKT inhibitor and/or an inhibitor of a PH domain protein, immune cells or checkpoint inhibitor may be administered sequentially or concurrently. Preferably, the first dose of AKT inhibitor and/or an inhibitor of a PH domain protein is administered concurrently with the administration of the immune cells and/or checkpoint inhibitor.

In an embodiment, the method provides for increased CD4+ and/or CD8+ CAR-T-cells in the spleen. In another embodiment, the method produces a smaller percentage of regulatory (T_{REG}) T-cells when compared to T-cells that are not cultured in the presence of an AKT inhibitor and/or an inhibitor of a PH domain protein and not administered with an AKT inhibitor and/or an inhibitor of a PH domain protein. In another embodiment, tumour T_{REG} cells are reduced by about 50% when compared to T-cells that are not cultured in the presence of an AKT inhibitor and/or an inhibitor of a PH domain protein and not administered with an AKT inhibitor and/or an inhibitor of a PH domain protein.

In another aspect, there is provided use of a population of immune cells, preferably T-cells comprising a chimeric antigen receptor (CAR-T-cells) and an AKT inhibitor and/or an inhibitor of a PH domain protein in the preparation of a medicament for modifying an immune response in a subject, preferably a T-cell immune response, wherein the immune cells were produced using a method comprising culturing immune cells in medium comprising an AKT inhibitor and/or an inhibitor of a PH domain protein.

In another aspect, there is provided use of a population of immune cells, preferably T-cells comprising a chimeric antigen receptor (CAR-T-cells) in the preparation of a medicament for modifying an immune response in a subject, preferably a T-cell response in a subject, wherein the subject has been, or will be, administered with AKT inhibitor and/or an inhibitor of a PH domain protein, wherein the immune cells were produced using a method comprising culturing immune cells in medium comprising an AKT inhibitor and/or an inhibitor of a PH domain protein

In another aspect, there is provided use of an AKT inhibitor and/or an inhibitor of a PH domain protein in the preparation of a medicament for modifying an immune response in a subject, preferably a T-cell response in a subject, wherein the subject has been, or will be, administered with of a population of immune cells, preferably T-cells comprising a chimeric antigen receptor (CAR-T-cells), wherein the immune cells were produced using a method comprising culturing immune cells in medium comprising an AKT inhibitor and/or an inhibitor of a PH domain protein.

In another aspect, there is provided a population of immune cells, preferably T-cells comprising a chimeric antigen receptor (CAR-T-cells) and an AKT inhibitor and/or an inhibitor of a PH domain protein for use in modifying an immune response in a subject, preferably a T-cell immune response, wherein the immune cells were produced using a method comprising culturing immune cells in medium comprising an AKT inhibitor and/or an inhibitor of a PH domain protein.

Also provided is a population of immune cells, preferably comprising T-cells comprising a chimeric antigen receptor (CAR-T-cells) for use in modifying an immune response in a subject, preferably a T-cell response, wherein the subject will be or has been administered with an AKT inhibitor and/or an inhibitor of a PH domain protein at least 18 hours before the medicament.

Also provided is an AKT inhibitor and/or an inhibitor of a PH domain protein for modifying a dendritic cell and/or natural killer cell response in a subject.

Also provided is the use of a population of cells comprising dendritic cells and/or natural killer cells for modifying a dendritic cell and/or natural killer cell response in a subject, wherein the subject has been, or will be, administered with an AKT inhibitor and/or an inhibitor of a PH domain protein.

In another aspect, the present invention provides a method of producing a cell population comprising immune cells, the method comprising culturing immune cells in medium comprising an AKT inhibitor and/or an inhibitor of a PH domain protein, preferably wherein the immune cells are T-cells, dendritic cells, natural killer cells, macrophages, myeloid cells or a combination thereof.

In an embodiment, the immune cells are transgenic. In an embodiment, the immune cells comprise a chimeric antigen receptor. In an embodiment, the immune cells are T-cells comprising a chimeric antigen receptor (CAR-T-cells).

In an embodiment, the method comprises
a) producing a T-cell enriched population of cells from a population of immune cells isolated from a subject;
b) transforming the T-cell enriched population of cells with a vector encoding a chimeric T-cell receptor: and
c) culturing the cells obtained in step b) in medium comprising an AKT inhibitor and/or an inhibitor of a PH domain protein.

In an embodiment, the CAR-T-cells produced using the method comprises central memory (T_{CM}) and/or stem cell (T_{SCM}) T-cells.

In an embodiment, at least about 10% of the CAR-T-cells produced using the method are T_{CM} and/or T_{SCM} cells. In an embodiment, at least about 15% of the CAR-T-cells produced using the method are T_{CM} and/or T_{SCM} cells. In an embodiment, at least about 20% of the CAR-T-cells produced using the method are T_{CM} and/or T_{SCM} cells. In an embodiment, at least about 25% of the CAR-T-cells produced using the method are T_{CM} and/or T_{SCM} cells. In an embodiment, at least about 25% of the CAR-T-cells produced using the method are T_{CM} and/or T_{SCM} cells. In an embodiment, between about 10% and about 60% of the CAR-T-cells produced using the method are T_{CM} and/or T_{SCM} cells. In an embodiment, between about 10% and about 50% of the CAR-T-cells produced using the method are T_{CM} and/or T_{SCM} cells. In an embodiment, between about 10% and about 40% of the CAR-T-cells produced using the method are T_{CM} and/or T_{SCM} cells. In an embodiment, between about 10% and about 30% of the CAR-T-cells produced using the method are T_{CM} and/or T_{SCM} cells.

In an embodiment, at least about 0.8% of the CD8+ T-cells produced using the method are T_{CM} and/or T_{SCM} cells. In an embodiment, at least about 1.5% of the CD8+ T-cells produced using the method are T_{CM} and/or T_{SCM} cells. In an embodiment, at least about 2.5% of the CD8+ T-cells produced using the method are T_{CM} and/or T_{SCM} cells. In an embodiment, at least about 3.5% of the CD8+ T-cells produced using the method are T_{CM} and/or T_{SCM} cells. In an embodiment, at least about 4.5% of the CD8+ T-cells produced using the method are T_{CM} and/or T_{SCM} cells. In an embodiment, between about 0.8% and about 15% of the CD8+ T-cells produced using the method are T_{CM} and/or T_{SCM} cells. In an embodiment, between about 0.8% and about 10% of the CD8+ T-cells produced using the method are T_{CM} and/or T_{SCM} cells. In an embodiment, between about 0.8% and about 5% of the CD8+ T-cells produced using the method are T_{CM} and/or T_{SCM} cells.

In an embodiment, at least about 0.37% of the total lymphocytes produced using the method are T_{CM} and/or T_{SCM} cells. In an embodiment, at least about 1% of the total lymphocytes produced using the method are T_{CM} and/or T_{SCM} cells. In an embodiment, at least about 2% of the total lymphocytes produced using the method are T_{CM} and/or T_{SCM} cells. In an embodiment, at least about 3% of the total lymphocytes produced using the method are T_{CM} and/or T_{SCM} cells. In an embodiment, at least about 4% of the total lymphocytes produced using the method are T_{CM} and/or T_{SCM} cells. In an embodiment, at least about 5% of the total lymphocytes produced using the method are T_{CM} and/or T_{SCM} cells. In an embodiment, between about 0.37% and about 15% of the total lymphocytes produced using the method are T_{CM} and/or T_{SCM} cells. In an embodiment, between about 0.37% and about 10% of the total lymphocytes produced using the method are T_{CM} and/or T_{SCM} cells. In an embodiment, between about 0.37% and about 5% of the total lymphocytes produced using the method are T_{CM} and/or T_{SCM} cells.

In an embodiment, the T_{CM} cells include CD45RO+ CD62L+ T-cells, preferably CD45RO+ CD62L^{hi} T-cells.

In an embodiment, the T_{SCM} cells include CD27⁺ CD95+ T-cells.

In an embodiment, the method further comprises enriching the cultured cells for the T_{CM} and/or T_{SCM} cells. Methods of selecting such cells from a population of cells are known in the art such as using antibody based cell sorting.

In an embodiment, the method produces a greater percentage of T_{CM} and/or T_{SCM} than T-cells cultured under identical conditions in the absence of the AKT inhibitor and/or an inhibitor of a PH domain protein.

In an embodiment, the method produces a smaller percentage of regulatory (T_{REG}) T-cells than T-cells cultured under identical conditions in the absence of the AKT inhibitor and/or an inhibitor of a PH domain protein.

In an embodiment, the T_{REG} cells are CD3+ CD4+ CD25+ FoxP3+ T-cells.

In an embodiment, the method produces a population of cells that express less of one or more inflammatory cytokines than if the same cells are cultured under identical conditions in the absence of the AKT inhibitor and/or an inhibitor of a PH domain protein. In an embodiment, the one or more inflammatory cytokines are TNFα, IFNγ or both.

In an embodiment, the method produces a greater percentage of naïve T-cells than T-cells cultured under identical conditions in the absence of the AKT inhibitor and/or an inhibitor of a PH domain protein.

The present inventors have determined that the methods of the invention can be used to produce improved CAR-T-cells target a viral infection. Thus, in an embodiment, the chimeric antigen receptor binds a viral antigen. In an embodiment, the method produces a population of CAR-T-cells with greater anti-viral activity than a population of CAR-T-cells cultured under identical conditions in the absence of the AKT inhibitor and/or an inhibitor of a PH domain protein.

In an alternate embodiment, the T-cells are not transgenic and have greater anti-viral activity than a population of T-cells cultured under identical conditions in the absence of the AKT inhibitor and/or an inhibitor of a PH domain protein.

In another embodiment, the method comprises
a) producing a dendritic cell enriched population of cells from a population of immune cells isolated from a subject;
b) exposing the cells from step a) to an antigen, and
c) culturing the cells in medium comprising an AKT inhibitor and/or an inhibitor of a PH domain protein. Thus, the method of the invention can be used to produce a dendritic cell vaccine. In an embodiment, the antigen is a cancer antigen or an antigen of a pathogen such as a viral antigen.

In an embodiment, the method produces more dendritic cells than dendritic cells cultured under identical conditions in the absence of the AKT inhibitor and/or an inhibitor of a PH domain protein.

In a further embodiment, the method comprises
a) producing a natural killer cell (NK) enriched population of cells from a population of immune cells isolated from a subject;
b) culturing the cells from step a) in medium comprising an AKT inhibitor and/or an inhibitor of a PH domain protein.

In an embodiment, the method produces a population of NK cells which have greater cytotoxic activity than a population of NK cells cultured under identical conditions in the absence of the AKT inhibitor and/or an inhibitor of a PH domain protein.

In an embodiment, the NK cells comprise a chimeric antigen receptor, and the method further comprises transforming the natural killer cell (NK) enriched population of cells with a vector encoding a chimeric antigen receptor.

In an embodiment, the concentration of the AKT inhibitor and/or an inhibitor of a PH domain protein in the culture medium is between about 0.5 μM and 9 μM, between about 1 μM and about 7 μM, between about 1 μM and about 5 μM, or between about 1 μM and 3 μM. In another embodiment, the concentration of the AKT inhibitor and/or an inhibitor of a PH domain protein in the culture medium is about 2 μM, about 3 μM, about 4 μM, about 5 μM, about 6 μM, about 7 μM, about 8 μM or about 9 μM.

In an embodiment, the cells are animal cells. In an embodiment, the cells are mammalian cells. In an embodiment, the cells are human cells.

In an embodiment, the cultured cells, or a sub-population thereof comprising the immune cells (such as which has been further enriched for a specific cell type(s) of interest), are administered to the subject.

In another aspect, the present invention provides population of cells produced using a method of the invention, preferably wherein the immune cells were produced using a method comprising culturing immune cells in medium comprising an AKT inhibitor and/or an inhibitor of a PH domain protein.

In a further aspect, the present invention provides a cell population comprising CAR-T-cells, wherein at least 10% of the CAR-T-cells are CD8+T_{CM} and/or T_{SCM} cells.

In an embodiment, the cell population has not been sorted, such as not sorted following culturing.

In an embodiment, less than 25% of the CAR-T-cells are TREGS.

In an embodiment, the immune cell population, preferably a population of T-cells comprising a chimeric antigen receptor (CAR-T-cells) of the invention, the AKT inhibitor and/or an inhibitor of a PH domain protein and/or the checkpoint inhibitor are administered in the form of a pharmaceutical composition. In an aspect, there is therefore provided is a pharmaceutical composition comprising the population of immune cells of the invention.

In an embodiment, the pharmaceutical composition comprises a population of immune cells, preferably T-cells comprising a chimeric antigen receptor (CAR-T-cells) of the invention and an AKT inhibitor and/or an inhibitor of a PH domain protein. In an embodiment, the pharmaceutical composition comprises a population of immune cells, preferably T-cells comprising a chimeric antigen receptor (CAR-T-cells) of the invention and a checkpoint inhibitor.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1—The impact of varying TCN and TCN-P concentrations on the viability of murine T-cells.

FIG. 2—Impact of repeated TCN exposure on proliferation of murine CAR-T-cells post-transduction.

FIG. 3—Impact of repeated TCN exposure on central memory phenotype (CD44+ CD62L^{hi}) in murine CD8+ CAR-T-cells.

FIG. 4—Impact of TCN treatment on IFNγ and TNFα production by murine CD8+ CAR-T-cells. CAR-T-cells pre-treated with TCN exposure produce comparable levels of IFNγ at 2:1 and 1: 1 effector: target ratios (A). A similar trend was observed with the release of TNFα at the same effector: target ratios (B).

FIG. 5—Impact of repeated TCN exposure on expression of early activation markers in murine CAR-T-cells in the absence of exposure to tumour antigens. TCN pre-treatment increased the expression of PD-1 (A) and CD69 (B) on the surfaces of murine CD8+ CAR-T-cells.

FIG. 6—Murine CAR-T-cells pre-treated with TCN exert blunted tumour antigen directed cytotoxicity (A) but TCN pre-treatment does not influence continued survival of CAR-T-cells.

FIG. 7—CAR-T transduction efficiency (Her2^{PE}) was unaltered in CD4 and CD8 T-cells.

FIG. 8—Flow cytometry contour plots showing the shift towards central memory phenotypes when CD8+ CAR-T-cells were treated with either TCN or TCN-P.

FIG. 9—Quantified flow cytometry data derived from FIG. 8 (24 hrs). Central memory T (T_{CM}) cell phenotypes (CD45RO+ CD62L+) increased in CD8+ CAR-T-cells from a mean of 11% (control, vehicle) to 17% (TCN, TCN-P), with a concomitant reduction of effector T (T_{E}) cells (8% vs 5%, n=3) (A). This pattern of response to TCN/TCN-P treatment was conserved across all 3 donors (B).

FIG. 10—The short exposure (24 hours) of CAR-T-cells to TCN or TCN-P resulted in a sustained shift of effector T (T_{E}) cells to central memory T (T_{CM}) cells for at least 3 days. This was consistent across all 3 PBMC donors.

FIG. 11—Quantified flow cytometry data derived from FIG. 10 (3 days). After the 24 hour treatment period, central memory T (T_{CM}) cell phenotypes (CD45RO+ CD62L+) remained increased in CD8+ CAR-T-cells (9% vs 16%, control/vehicle vs TCN/TCN-P), which was concomitant with a reduction in effector T (T_{E}) cells (35% vs 23%, n=3) (A). This pattern of response to TCN/TCN-P treatment was conserved across all 3 donors (B).

FIG. 12—Pre-treatment with TCN/TCN-P increased CCR7+ T_{CM} from a mean of 9% to 16%, concomitant with a reduction in CCR7+ T_{E} from 35% to 23%.

FIG. 13—Pretreatment with TCN or TCN-P did not have exert any effect on CD4 T_{CM} (CD45RO+ CD62L+) at 24 hrs post treatment.

FIG. 14—Quantified flow cytometry data derived from FIG. 14 (24 hrs), where pre-treatment with TCN/TCN-P did not affect CD4+ T_{CM} or CD4+ T_{E}.

FIG. 15—Pretreatment with TCN/TCN-P did not have exert any effect on CD4 T_{CM} (CD45RO+ CD62L+) at 24 hrs post treatment.

FIG. 16—Quantified flow cytometry data derived from FIG. 15 (3 days), where pre-treatment with TCN/TCN-P did not affect CD4+ T_{CM} or CD4+ T_{E}.

FIG. 17—TCN pre-treatment reduced regulatory T (T_{REG}) cell subpopulations (CD4+CD25+FoxP3+) in CAR-T-cells.

FIG. 18—Overview of protocol to measure in vivo effects of TCN or TCN-P pre-treatment on CAR-T-cell function.

FIG. 19—TCN-P pre-treatment during CAR-T manufacturing results in enrichment for central memory T-cells. Illustrative representation of E0771-hHer2 breast cancer model, where 200,000 tumour cells were orthotopically transplanted in the mammary fat pat and 20 million CAR-T-cells were administered via tail vein injection 6 days later. Animals were humanely culled when tumours were >120 mm² or when other humane endpoints were reached (A). Representative flow cytometry contour plots showing that TCN-P preconditioning had no impact on the CD4:CD8 T-cell ratio during the manufacturing process (B, upper panel), but resulted in preferential enrichment for CD44+ CD62L+ central memory CD8+ T-cells (B, lower panel). Animals received the same proportions of CD4:CD8 T CAR+ cells (C). Administration of untreated CAR-T-cells reduced tumour volume and extended the survival of animals by two days, however tumour control was most significantly reduced by TCN-P preconditioned CAR-T-cells. Survival was also increased by 14 days (D). This translated to a significant improvement to the probability of survival (E, p=0.0012).

FIG. 20—Reductions in tumour growth corresponded to sustained central memory phenotype post CAR-T administration. A separate cohort of animals were subjected to the same breast tumour model and all animals were sacrificed on day 8 post CAR-T treatment, with tumour measurements taken every 2-3 days (A). Representative flow cytometric analysis showed that preconditioning with TCN-P could increase the proportion of circulating CD4+ T-cells compared to CD8+ T-cells (B, upper panel), reduce CD8+ CAR-T-cells (middle panel), while sustaining the central memory phenotype (lower panel). Intratumoural CD4+ and CD8+ T-cells were not significantly altered (C), however, greater numbers of intrasplenic CD4+ T-cells were observed in the preconditioned group (D). The changes to tumour control were not associated with significant changes to IFN-gamma and TNF-alpha production (E).

FIG. 21—Impact of TCN-P preconditioning on circulating T-cells. Preconditioning did not have significant impact on CD4+ or CD8+ T-cell numbers (A-B). No significant changes were observed in CD8+ CAR-T-cells (C) but the number of CD4+ CAR-T were significantly higher in the animals that received preconditioned CAR-T-cells (D, p=0.0009). The percentage of CD8+ and CD4+ central memory T-cells were higher in animal that received preconditioned CAR-T-cells (E-F, p<0.0001). This was concurrent with reduced CD8+(G, p<0.0001) and CD4+(H, p=0.0029) effector memory T-cells.

FIG. 22—Impact of TCN-P preconditioning on intratumoral and intrasplenic T-cells. No significant changes were observed in intratumoural and intrasplenic percentages of CD4+ and CD8+ central memory T-cells (A, C). No changes were observed in intratumoural percentages of CD101+CD8+ T-cells (B), while intrasplenic levels of CD101+CD8+ T-cells were lower in the preconditioned group, suggesting that preconditioning conferred a protective effect against exhaustion in CD8+ T-cells (D).

FIG. 23—TCN-P preconditioned CAR-T-cells in combination with PD-1 checkpoint blockade improves anti-tumour efficacy against solid tumour. A) Her2+ transgenic mice were inoculated with 2.5^{e5} E0771-hHer2. Mice were randomised 5 days post-tumour inoculation to have an average tumour size of 20 mm². They were either left as non-treated or treated with therapies: anti-PD-1 (aPD1), CAR-T-cell (CAR-T-cells+2A3), CAR-T-cells with anti-PD-1 (CAR-T+aPD1), TCN-P pre-conditioned CAR-T-cells (PTX-2+2A3) and TCN-P pre-conditioned CAR-T-cells combined with anti-PD-1 checkpoint blockade (PTX-2+aPD1). Tumour growth was measured every 2-3 days (mm²). Each treatment group is consisted of 6 mice. B) Survival curve of mice in these treatments.

FIG. 24—Overview of protocol to measure in vivo efficacy of TCN and TCN-P as a neoadjuvant for CAR-T.

FIG. 25—The adjuvant potency of TCN-P. Her2+ transgenic mice were inoculated with 2.5^{e5} MC-38-hHer2. Mice were randomised 5 days post-tumour inoculation to have an average tumour size of 20 mm². As treatment, they were intraperitoneally injected with either DMSO (vehicle control) or TCN-P at 5, 25 or 50 mg/Kg every 3-4 days. Tumour growth was measured every 2-3 days (mm²). Each treatment group is consisted of 6 mice. TCN-P has a dosage dependent anti-tumour effect.

FIG. 26—TCN-P enhances CAR-T-cell therapy efficacy against solid tumour. A) Her2+ transgenic mice were inoculated with 2.5^{e5} MC-38-hHer2. They were randomised 5 days post-tumour inoculation to have an average tumour size of 20 mm². They were either left as non-treated or subject to a treatment regime including interperitoneal injection with TCN-P (25 mg/Kg) every 3-4 days: intravenous injection with 20^{e6} anti-human Her2 CAR-T-cells and the combination treatment of intravenous injection with 20×10⁶ anti-human Her2 CAR-T-cells with TCN-P at 25 mg/Kg (injected every 3-4 days). Tumour growth is measured every 2-3 days (mm²). Each treatment group is consisted of 6 mice. B) Tumour growth of individual mice (N=6/treatment group). C) TCN-P enhances the survival of mice bearing solid MC-38-hHer2 tumour. Tumour-bearing mice received the combined therapy of CAR-T-cells and TCN-P (25 mg/Kg) showed increased median survival by 6 days compared to treatment with only CAR-T-cells.

FIG. 27—TCN-P as an adjuvant for CAR-T treatment. Illustrative representation of the MC-38 colon cancer model where 250,000 MC-38 tumour cells are transplanted subcutaneously approximately 6 days prior to commencement of CAR-T treatments and adjuvant TCN-P co-administration every 3 days (A). Preliminary data indicate that TCN-P adjuvant therapy significantly improved CAR-T directed tumour reduction (*p<0.05), but the most effective regimen was the combination of TCN-P adjuvant and TCN-P preconditioned CAR-T-cells.

FIG. 28—A) The combination of TCN-P pretreatment and TCN-P adjuvant therapy resulted in a significant accumulation and/or expansion of CD4+ and CD8+ CAR T-cells in the spleen (p<0.0001) by day 14 post treatment. B) Administration of TCN-P resulted in a 50% reduction of intratumoral Tregs.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, CAR-T technology, immunology, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A: X employs B: or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

As used herein, the term “subject” can be any animal. In one embodiment, the animal is a vertebrate. For example, the animal can be a mammal, avian, chordate, amphibian or reptile. Exemplary subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one embodiment, the mammal is a human. In an embodiment, a method of the invention is for veterinary use.

The terms “treatment” or “treating” of a subject includes the application or administration of a population of immune cells of the invention, or a composition thereof for the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement: remission; lessening of the rate of worsening: lessening severity of the disease: stabilization, diminishing of symptoms or making the injury, pathology or condition more tolerable to the subject: slowing in the rate of degeneration or decline: making the final point of degeneration less debilitating: or improving a subject's physical or mental well-being.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians. For example, in the case of a subject suspected of having breast cancer, a subject may have a family history of the cancer and has been identified as having a mutation that is likely to give rise to the cancer, but does not yet show any apparent symptoms of the disease. In this case, it is contemplated that the immune cells of the invention, or a composition thereof will have utility in the preventing the onset of one or more symptoms associated with disease (e.g., breast cancer) in the subject.

As used herein, “cytokine release syndrome” (CRS) refers to an acute systemic inflammatory syndrome characterized by fever and multiple organ dysfunction that is associated with chimeric antigen receptor (CAR)-T-cell therapy, therapeutic antibodies, and haploidentical allogeneic transplantation

As used herein, an “enriched population” or variations thereof refers to a population of cells that have had been processed to remove, or at least reduce the representation of, some types of cells from the starting population of cells, such as peripheral blood mononuclear cell (PBMCs) population isolated from a subject. Methods of positively or negatively selecting specific cell types are well known in the art, such as using magnetic beads comprising antibodies that selectively bind cell surface proteins of a specific cell type to be enriched or removed. In an embodiment, when compared to the starting cell population (such as PBMCs), the cell type(s) for which the population has been enriched has, for example, a 1.5 fold, 2 fold, 5 fold, 10 fold, 20 fold or 50 fold higher representation in the enriched population when compared to the starting population.

As used herein, the term “identical conditions” is a relative term which means that the same cell population, for example split into two identical sub-populations, when exposed to the exact same culturing procedure apart from the presence of the inhibitor for one of the sub-populations.

The terms “combination therapy”, “administered in combination” or “co-administration” or the like, as used herein, are meant to encompass administration of the selected therapeutic agents to a single subject, and are intended to include treatment regimens in which the agents are administered by the same or different route of administration or at the same or different time.

### Inhibitors

As used herein, the term “PH domain protein” refers to a protein comprising a PH domain. Pleckstrin homology domain (PH domain) or (PHIP) is a protein domain of approximately 100-120 amino acids that occurs in numerous proteins involved in intracellular signalling or as constituents of the cytoskeleton. All share the same β-sandwich fold first observed in NMR structures of the N-terminal pleckstrin PH domain. The amino-terminal half of the protein forms a four-stranded β-sheet, with an additional short α-helix in the β3/β4 loop (specific to the β-spectrin PH domain). The second half of the protein forms a B-sheet meander (strands β5-β7) that is near-orthogonal to the first sheet. The two sheets form a ‘sandwich’ that is filled with the hydrophobic core of the domain. In one embodiment, the PH domain protein is a small G protein. In another embodiment, the PH domain protein is a serine/threonine-specific protein kinase. In another embodiment, the PH domain protein is an oxysterol-binding proteins (OSBP). In another embodiment, the PH domain protein is a G protein receptor kinase. Examples of PH domain protein which can be inhibited using a method of the invention include, but are not limited to, Oxysterol-binding protein 1, Oxysterol-binding protein 2, Spectrin beta chain, non-erythrocytic 1, Rho GTPase-activating protein 27, Phosphoinositide 3-kinase (PI3K), AKT, or a combination of one or more thereof. Examples of inhibitors of a PH domain protein useful for the invention include, but are not limited to, phosphatidylinositol ether lipid analogs (PIAs), such as D-3-deoxy-myo-inositols, for example, D-3-deoxy-phosphatidyl-myo-inositol 1-[(R)-2-methoxy-3-octadecyloxypropyl hydrogen phosphate] (DPIEL, PX-316): alkyl-phospholipids (APLs), such as edelfosine, miltefosine, and perifosine: inositol phosphates (IPs), such as Ins (1,3,4,5,6)pentakisphosphate (IP5), Ins (1,4,5,6) tetrakisphosphate (IP4), phytic acid (IP6), 2-O-benzy-myo-inositol1,3,4,5,6-pentakisphosphate (2-O-Bn-InsPs), diphosphoinositol pentakisphosphate (5-PP-IP5): inositolphosphate-6-kinasel (IP6K1): sulphonamides, such as diazo-sulpho-amido inhibitors, for example NSC348900 (PH-316) and 4-Dodecyl-N-(1,3,4-thiadiazol-2-yl)benzenesulfonamide (for example PH-427): purine/pyrimidine analogs, such as triciribine (tricyclic dinucleoside, NSC 154020, TCN, AKT/PKB signaling Inhibitor-2, API-2), triciribine phosphate (NSC 280594: triciribine 5′-monophosphate: TCN-P), API-1 (NSC 177223-Pyrido[2,3-d]pyrimidines): and other inhibitors such as allosteric compounds that interact only within the PH domain via Trp80 (for example MK-2206, SC66), tirucallic acids, PITenins (PITs), peptide mimetics (for example AKT-ins, such as NH2-AVTDHPDRLWAWEKF—COOH), 1,2,3-triazol-4-yl methanol-based antagonists: and salts, esters, analogues, variants, and derivatives thereof. In an embodiment, the PH domain protein inhibitor is triciribine (TCN) or triciribine 5′-monophosphate (TCN-P).

AKT, also known as protein kinase B (PKB), is a serine/threonine-specific protein kinase that plays a key role in multiple cellular processes such as glucose metabolism, apoptosis, cell proliferation, transcription, and cell migration. AKTI is involved in the PI3K/AKT/mTOR pathway and other signalling pathways. Examples of AKT inhibitors for use in the invention include, but are not limited to, MK-2206 2HCI (8-[4-(1-Aminocyclobutyl)phenyl]-9-phenyl[1,2,4]triazolo[3,4-f][1,6]napht-hyridin-3(2H)-one dihydrochloride): perifosine (1,1-dimethyl-4 [(octadecyloxy)hydroxyphosphinyl]oxy]-piperidinium inner salt, KRX-0401): GSK690693 (4-[2-(4-amino-1,2,5-oxadiazol-3-yl)-1-ethyl-7-[[(3 S)-piperidin-3-yl]methoxy]imidazo[4,5-c]pyridin-4-yl]-2-methylbut-3-yn-2-ol): ipatasertib ((2S)-2-(4-Chlorophenyl)-1-{4-[(5R,7R)-7-hydroxy-5-methyl-6,7-dihydro-5H-cyclopenta[d]pyrimidin-4-yl]-1-piperazinyl}-3-(isopropylamino)-1-propanone-, GDC-0068): AZD5363 (4-amino-N-[(1S)-1-(4-chlorophenyl)-3-hydroxypropyl]-1-(7H-pyrrolo[2,3-d]-pyrimidin-4-yl)piperidine-4-carboxamide): PF-04691502 (2-amino-8-[4-(2-hydroxyethoxy)cyclohexyl]-6-(6-methoxypyridin-3-yl)-4-me-thylpyrido[2,3-d]pyrimidin-7-one): AT7867 (4-(4-chlorophenyl)-4-[4-(1H-pyrazol-4-yl)phenyl]piperidine): Triciribine (5-Methyl-1-(β-D-ribofuranosyl)-1,5-dihydro-1,4,5,6,8-pentaazaacenap-hthylen-3-amine); triciribine 5′-monophosphate: CCT128930 (4-(4-Chlorobenzyl)-1-(7H-pyrrolo[2,3-d]pyrimidin-4-yl)-4-piperidinamine)-: A-674563 ((2S)-1-[5-(3-methyl-2H-indazol-5-yl)pyridin-3-yl]oxy-3-phenylpropan-2-am-ine): PHT-427 (4-dodecyl-N-(1,3,4-thiadiazol-2-yl)benzenesulfonamide): AKTi-1/2 (3-[1-[[4-(7-phenyl-3H-imidazo[4,5-g]quinoxalin-6-yl)phenyl]methyl]piperi-din-4-yl]-1H-benzimidazol-2-one): afuresertib (GSK2110183, N-[(2 S)-1-amino-3-(3-fluorophenyl)propan-2-yl]-5-chloro-4-(4-chloro-2-methylpy-razol-3-yl)thiophene-2-carboxamide): AT13148 ((1S)-2-amino-1-(4-chlorophenyl)-1-[4-(1H-pyrazol-4-yl)phenyl]ethanol): Miltefosine (hexadecyl 2-(trimethylazaniumyl)ethyl phosphate): Honokiol (2-(4-hydroxy-3-prop-2-enylphenyl)-4-prop-2-enylphenol): TIC10 Analogue (2,6,7,8,9,10-hexahydro-10-[(2-methylphenyl)methyl]-7-(phenylmethyl)-imid-azo[1,2-a]pyrido[4,3-d]pyrimidin-5(3H)-one); AKT inhibitor VIII (1,3-Dihydro-1-(1-((4-(6-phenyl-1H-imidazo[4,5-g]quinoxalin-7-yl)phenyl)methyl)-4-piperidinyl)-2H-benzimidazol-2-one): Uprosertib (GSK2141795): TIC10 (2,4,6,7,8,9-hexahydro-4-[(2-methylphenyl)methyl]-7-(phenylmethyl)-imidaz-o[1,2-a]pyrido[3,4-e]pyrimidin-5(1H)-one): Capivasertib (AZD5363) and MS-222 (ethyl-3-aminobenzoate methanesulfonate salt), and salts, esters, analogues, variants, and derivatives thereof. In an embodiment, the AKT inhibitor is triciribine, triciribine 5′-monophosphate, AKT inhibitor VIII, MK-2206, AZD5363, GDC-0068, GSK2141795 and GSK2110183 hydrochloride, and salts, esters, analogues, variants, and derivatives thereof. In an embodiment, the AKT inhibitor is triciribine (TCN) or triciribine 5′-monophosphate (TCN-P).

Inhibition of the activity of AKT and/or a PH domain protein may be less than 100%, e.g., about 10% to about 95%, e.g., about 20%, about 30%, about 40%, about 50%, about 60%, about 70%, about 80%, about 90%, or another percent inhibition of the activity from, for example, about 10% to about 95%. The inhibitor may be, for example, a small molecule, a peptide, a protein (such as an antibody), a nucleic acid, or a combination thereof.

### Immune Cells

As used herein, the phrase “immune cell” refers to a cell which is capable of affecting or inducing an immune response upon recognition of an antigen. In some embodiments, the immune cell is a T-cell, a natural killer (NK) cell, a macrophage, a myeloid cell or a dendritic cell. In some embodiments, the cell is a mammalian cell. In some embodiments, the cell is a human cell. The cells may be autologous or allogeneic to the subject to which they are administered. In an embodiment, the present invention provides a population of CAR-expressing cells such as CAR-T-cells.

As used herein, the phrase “modifying an immune response” or modifying a T-cell immune response” refers to an ability of an immune cell, or T-cell to induce or increase an immune response upon recognition of an antigen. Such modification of an immune response or T-cell response will be understood to be sufficient for the treatment of a cancer, an infection, or an inflammatory disease as described herein.

As used herein, the phrase “cytotoxicity activity” refers to the ability of an immune cell, such as an NK cell, to destroy living cells.

As used herein, the term “immune response” has its ordinary meaning in the art, and includes both humoral and cellular immunity. An immune response can manifest as one or more of, the development of anti-antigen antibodies, expansion of antigen-specific T-cells, increase in tumor infiltrating-lymphocytes (TILs), development of an anti-tumor or anti-tumor antigen delayed-type hypersensitivity (DTH) response, clearance of the pathogen, suppression of pathogen and/or tumor growth and/or spread, tumor reduction, reduction or elimination of metastases, increased time to relapse, increased time of pathogen or tumor free survival, and increased time of survival. An immune response may be mediated by one or more of, B-cell activation, T-cell activation, natural killer cell activation, activation of antigen presenting cells (e.g., B cells, DCs, monocytes and/or macrophages), cytokine production, chemokine production, specific cell surface marker expression, in particular, expression of co-stimulatory molecules. The immune response may be characterized by a humoral, cellular, Th1 or Th2 response, or combinations thereof. In an embodiment, the immune response is an innate immune response.

### T-Cells

In some embodiments, the immune cell is a T-cell e.g. a CAR-T-cell. T-cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are several subsets of T-cells, each with a distinct function.

In an embodiment, the T-cells are or include central memory (T_{CM}) T-cells. T_{CM} cells patrol lymph nodes, providing central immunosurveillance against known pathogens, but have not been described as conducting primary tissue immunosurveillance. In an embodiment, T_{CM} cells produced using a method of the invention include CD45RO+ CD62L+ T-cells, preferably CD45RO+ CD62L^{hi} T-cells. Such cells may also be CCR7+.

In an embodiment, the T-cells are or include central memory stem cell (T_{SCM}) T-cells. T_{SCM} cells a rare subset of memory lymphocytes endowed with the stem cell-like ability to self-renew and the multipotent capacity to reconstitute the entire spectrum of memory and effector subset. In an embodiment, the T_{SCM} cells include CD27⁺CD95⁺ T-cells.

In an embodiment, the method produces a greater percentage of Tom and/or T_{SCM} than T-cells cultured under identical conditions in the absence of the AKT inhibitor and/or an inhibitor of a PH domain protein. In an embodiment, the method produces at least about 25% more central memory (T_{CM}) and/or stem cell (T_{SCM}) T-cells than T-cells cultured under identical conditions in the absence of the AKT inhibitor and/or an inhibitor of a PH domain protein. In an embodiment, the method produces at least about 50% more central memory (T_{CM}) and/or stem cell (T_{SCM}) T-cells than T-cells cultured under identical conditions in the absence of the AKT inhibitor and/or an inhibitor of a PH domain protein. In an embodiment, the method produces at least about 75% more central memory (T_{CM}) and/or stem cell (T_{SCM}) T-cells than T-cells cultured under identical conditions in the absence of the AKT inhibitor and/or an inhibitor of a PH domain protein. In an embodiment, the method produces between about 25% and about 90% more central memory (Tc) and/or stem cell (T_{SCM}) T-cells than T-cells cultured under identical conditions in the absence of the AKT inhibitor and/or an inhibitor of a PH domain protein.

As used herein, a regulatory T-cell (T_{REG}), or variations thereof, refers to a population of T-cells which are crucial for the maintenance of immunological tolerance. Their major role is to shut down T-cell-mediated immunity toward the end of an immune reaction and to suppress auto-reactive T-cells that escaped the process of negative selection in the thymus. Two major classes of CD4+ T_{REG} cells have been described—Foxp3+ and Foxp3−.

In an embodiment, the method produces a smaller percentage of regulatory (T_{REG}) T-cells than T-cells cultured under identical conditions in the absence of the AKT inhibitor and/or an inhibitor of a PH domain protein. In an embodiment, the method produces at least about 5% less regulatory (T_{REG}) T-cells than T-cells cultured under identical conditions in the absence of the AKT inhibitor and/or an inhibitor of a PH domain protein. In an embodiment, the method produces at least about 10% less regulatory (T_{REG}) T-cells than T-cells cultured under identical conditions in the absence of the AKT inhibitor and/or an inhibitor of a PH domain protein. In an embodiment, the method produces at least about 15% less regulatory (T_{REG}) T-cells than T-cells cultured under identical conditions in the absence of the AKT inhibitor and/or an inhibitor of a PH domain protein. In an embodiment, the method produces at least about 20% less regulatory (T_{REG}) T-cells than T-cells cultured under identical conditions in the absence of the AKT inhibitor and/or an inhibitor of a PH domain protein. In an embodiment, the method produces at least about 25% less regulatory (T_{REG}) T-cells than T-cells cultured under identical conditions in the absence of the AKT inhibitor and/or an inhibitor of a PH domain protein. In an embodiment, the method produces between about 5% and about 30% less regulatory (T_{REG}) T-cells than T-cells cultured under identical conditions in the absence of the AKT inhibitor and/or an inhibitor of a PH domain protein. In an embodiment, the method produces between about 5% and about 25% less regulatory (T_{REG}) T-cells than T-cells cultured under identical conditions in the absence of the AKT inhibitor and/or an inhibitor of a PH domain protein. In an embodiment, the T_{REG} cells are CD25+ FoxP3+ T-cells.

As used herein, the term “naïve T-cells” refers to a population of T-cells that has matured and been released by the thymus but has not yet encountered its corresponding antigen. In other words, naïve T-cells are in the stage between maturity and activation. Naive T-cells are commonly characterized by the surface expression of L-selectin (CD62L) and C—C Chemokine receptor type 7 (CCR7): the absence of the activation markers CD25, CD44 or CD69; and the absence of memory CD45RO isoform. They also express functional IL-7 receptors, consisting of subunits IL-7 receptor-a, CD127, and common-y chain, CD132.

A T-cell lacking a functional endogenous T-cell receptor (TCR) can be, e.g., engineered such that it does not express any functional TCR on its surface, engineered such that it does not express one or more subunits that comprise a functional TCR or engineered such that it produces very little functional TCR on its surface. Alternatively, the T-cell can express a substantially impaired TCR, e.g., by expression of mutated or truncated forms of one or more of the subunits of the TCR. The term “substantially impaired TCR” means that this TCR will not elicit an adverse immune reaction in a host.

A T-cell described herein can be, e.g., engineered such that it does not express a functional HLA on its surface. For example, a T-cell described herein, can be engineered such that T-cell surface expression HLA, e.g., HLA class 1 and/or HLA class II, is downregulated. In some embodiments, the T-cell can lack a functional TCR and a functional HLA, e.g., HLA class I and/or HLA class II.

Modified T-cells that lack expression of a functional TCR and/or HLA can be obtained by any suitable means, including a knock out or knock down of one or more subunit of TCR or HLA. For example, the T-cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

### Natural Killer Cells

In some embodiments, the immune cell is a natural killer cell. Natural-killer (NK) cells are CD56 CD3 large granular lymphocytes that can kill infected and transformed cells, and constitute a critical cellular subset of the innate immune system. Unlike cytotoxic CD8+ T lymphocytes, NK cells launch cytotoxicity against tumour cells without the requirement for prior sensitization, and can also eradicate MHC-I-negative cells. In an embodiment, the NK cells are CD3−CD56+ CD7+CD127− NKp46+T-bet+Eomes+. In an embodiment, cytotoxic NK cells CD56^{dim} CD16+.

### Dendritic Cells

In some embodiments, the immune cell is a dendritic cell. Dendritic cells are a heterogeneous group of specialized antigen-presenting cells that originate in the bone marrow from CD34+ stem cells and express major histocompatibility complex (MHC) class II molecules. Mature dendritic cells are able to prime, activate and expand effector immune cells, such as T-cells and NK cells. Dendritic cell therapy is known in the art (see, e.g. Sabado et al., 2017). Briefly, dendritic cells can be isolated from a patient, exposed to a disease-specific antigen, for example a cancer specific antigen, or genetically modified to express a CAR, or a disease specific antigen, and are then infused back into the patient where they prime, activate and expand effector immune cells, for example T-cells.

### Myeloid Cells

In some embodiments, the immune cell is a myeloid cell. Granulocytes, monocytes, macrophages, and dendritic cells represent a subgroup of leukocytes, collectively called myeloid cells. They circulate through the blood and lymphatic system and are rapidly recruited to sites of tissue damage and infection via various chemokine receptors. Within the tissues they are activated for phagocytosis as well as secretion of inflammatory cytokines, thereby playing major roles in protective immunity. Myeloid cell therapies are known in the art and may be useful in the treatment of cancer, infection or disease. For instance, myeloid cells are known to be abundant in the tumour stroma and the presence of these cells may influence patient outcome in many cancer types. Briefly, myeloid cells can be isolated from a patient, exposed to a disease-specific antigen, for example a cancer specific antigen, or genetically modified to express a CAR, or a disease specific antigen, and are then infused back into the patient where they prime, activate and expand effector immune cells, for example T-cells.

### Macrophages

In some embodiments, the immune cell is a macrophage. Macrophages are myeloid lineage cells that arise from bone marrow derived monocytic progenitor cells that differentiate into tissue macrophages, antigen-presenting dendritic cells and bone resorbing osteoclasts. Macrophage cell therapies are known in the art and may be useful in the treatment of cancer, infection or disease. Briefly, macrophages can be isolated from a patient, exposed to a disease-specific antigen, for example a cancer specific antigen, or genetically modified to express a CAR, or a disease specific antigen, and are then infused back into the patient where they prime, activate and expand effector immune cells, for example T-cells.

### Chimeric Antigen Receptors

The term “chimeric antigen receptor” or alternatively “CAR” refers to a polypeptide or set of polypeptides, which when in an immune cell, provides the cell with specificity for a target T-cell, for example a cancer cell, and with intracellular signal generation.

CARs can be used to generate immune cells, such as T-cells, dendritic cells, or natural killer (NK) cells, specific for selected targets. Suitable constructs for generating CARs are described in U.S. Pat. No. 5,843,728: U.S. Pat. No. 5,851,828: U.S. Pat. No. 5,912,170: U.S. Pat. No. 6,004,811: U.S. Pat. No. 6,284,240: U.S. Pat. No. 6,392,013: U.S. Pat. No. 6,410,014: U.S. Pat. Nos. 6,753,162; 8,211,422; and WO9215322. Alternative CAR constructs can be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signalling domains of either CD3C or FcRy or scFv-FcRy (see, e.g., U.S. Pat. No. 7,741,465: U.S. Pat. No. 5,912,172: and U.S. Pat. No. 5,906,936). Second-generation CARs incorporate the intracellular domains of one or more costimulatory molecules, such as CD28, CD28z, OX40 (CD134), or 4-1BB (CD137) within the endodomain, e.g., scFv-CD28/OX40/4 BB-CD3 (see, e.g., U.S. Pat. No. 8,911,993: U.S. Pat. No. 8,916,381: U.S. Pat. No. 8,975,071: U.S. Pat. No. 9,101,584: U.S. Pat. No. 9,102,760: U.S. Pat. No. 9,102,761). Third-generation CARs include a combination of costimulatory endodomains, such a CD3C-chain, CD97, GDI la-CD18, CD2, ICOS, CD27, CD154, CDS, OX40, 4-1BB, or CD28 signalling domains, e.g., scFv-CD28-4 BB-CD3C or scFv-CD28-OX40-CD3Q (see, e.g., U.S. Pat. No. 8,906,682: U.S. Pat. No. 8,399,645: U.S. Pat. No. 5,686,281: WO2014134165; and WO2012079000). In some embodiments, costimulation can be coordinated by expressing CARs in antigen-specific T-cells, chosen so as to be activated and expanded following, for example, interaction with antigen on professional antigen-presenting cells, with costimulation. Additional engineered receptors can be provided on the immune cells, e.g., to improve targeting of a T-cell attack and/or minimize side effects.

### Methods of Preparing CAR-Expressing Cells

### Sources of Cells

Prior to expansion, and possible genetic modification or other modification, a cell population comprising or consisting of immune cells such as T-cells, dendritic cells, natural killer (NK) cells or a combination thereof, can be obtained from a subject. Immune cells can be obtained from a number of sources, including peripheral blood mononuclear cells, bone marrow, lymph node tissue, cord blood, thymus tissue, tissue from a site of infection, ascites, pleural effusion, spleen tissue, and tumours.

In certain embodiments of the present disclosure, immune cells, e.g., T-cells, can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T-cells, monocytes, granulocytes, B cells, dendritic cells, other nucleated white blood cells, red blood cells, and platelets. In one embodiment, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al. (2015).

In one embodiment, T-cells are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune cells, e.g., T-cells, that are a T regulatory cell-depleted population. A CD25+ depleted cell population, for example, can be obtained using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells. However, as discussed herein, the AKT inhibitors and/or an inhibitors of a PH domain protein used in the methods of the invention alone is able to reduce T_{REG} cells during culturing.

In one embodiment, T regulatory (T_{REG}) cells, e.g., CD25+ T-cells, are removed from the population using an anti-CD25 antibody, or fragment thereof, or a CD25− binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25− depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T-cell, NK cell) product.

In an embodiment, a method of the invention does not comprise sorting the cultured cells to isolate CD45RO− CCR7− CD62L− T memory cells.

T-cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T-cells, isolated and frozen for later use in immune cell therapy for any number of diseases or conditions that would benefit from immune cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T-cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, Cytoxan, fludarabine, cyclosporin, FK506, rapamycin, mycophenolic acid, steroids, FR901228, and irradiation.

### Methods of Making CAR-Expressing Cells

In an embodiment, a method of the invention includes the making CAR-expressing cells by introducing a vector or nucleic acid encoding a CAR into a cell. Methods of introducing and expressing genes into a cell are known in the art. In the context of an expression vector, the vector can be readily introduced into a host T-cell, e.g., mammalian, bacterial, yeast, or insect T-cell by any method in the art. For example, the expression vector can be transferred into a host T-cell by physical, chemical, or biological means.

Physical methods for introducing a polynucleotide into a host T-cell include calcium phosphate precipitation, lipofection, particle bombardment, microinjection, electroporation, and the like. Methods for producing cells comprising vectors and/or exogenous nucleic acids are well-known in the art (see, for example, Sambrook Molecular Cloning: A Laboratory Manual, volumes 1-4, Cold Spring Harbor Press). A preferred method for the introduction of a polynucleotide into a host T-cell is calcium phosphate transfection.

Biological methods for introducing a polynucleotide of interest into a host T-cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, for example, U.S. Pat. Nos. 5,350,674 and 5,585,362).

Chemical means for introducing a polynucleotide into a host T-cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

An exemplary non-viral delivery vehicle is a liposome. The use of lipid formulations is contemplated for the introduction of the nucleic acids into a host T-cell (in vitro, ex vivo or in vivo). In another embodiment, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma Aldrich: dicetyl phosphate (“DCP”) can be obtained from K & K Laboratories: cholesterol (“Choi”) can be obtained from Calbiochem-Behring: dimyristyl phosphatidylglycerol (“DMPG”) and other lipids may be obtained from Avanti Polar Lipids, Inc. for example. Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium.

Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes. Regardless of the method used to introduce exogenous nucleic acids into a host T-cell or otherwise expose a cell to the inhibitor of the present invention, in order to confirm the presence of the recombinant DNA sequence in the host T-cell, a variety of assays may be performed. Such assays include, for example, “molecular biological” assays well known to those of skill in the art, such as Southern and Northern blotting, RT-PCR and PCR: “biochemical” assays, such as detecting the presence or absence of a particular peptide, e.g., by immunological means (ELISAs and Western blots) or by assays described herein to identify agents falling within the scope of the invention.

### Methods of Culturing and Expanding Immune Cells

Immune cells such as T-cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and US 20060121005.

Expanding the T-cells by the methods disclosed herein can multiply the cells by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers there between. In one embodiment, the T-cells expand in the range of about 20 fold to about 50 fold.

In an embodiment, the cells are cultured for between about 7 days and about 14 days, or about 7 days to about 10 days.

Generally, a population of immune cells e.g., T regulatory cell depleted cells, may be expanded by contact with a surface having attached thereto an agent that stimulates a CD3/TCR complex associated signal and a ligand that stimulates a costimulatory molecule on the surface of the T-cells. In particular, T-cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T-cells, a ligand that binds the accessory molecule is used. For example, a population of T-cells can be contacted with an anti-CD3 antibody and an anti-CD28 antibody, under conditions appropriate for stimulating proliferation of the T-cells. To stimulate proliferation of either CD4+ T-cells or CD8+ T-cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., 1998: Haanen et al., 1999; Garland et al., 1999).

Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGF, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T-cells. Antibiotics, e.g., penicillin and streptomycin, are included only in experimental cultures, not in cultures of cells that are to be infused into a subject. The target T-cells are maintained under conditions necessary to support growth, for example, an appropriate temperature (e.g., 37° C.) and atmosphere (e.g., air plus 5% CO2).

### CAR-Expressing Cell Therapy

### CAR-T-Cell Therapy

CAR-T-cell therapy is a type of cellular therapy where immune cells (e.g., T-cells) are genetically modified to express a CAR and the CAR-expressing cell (e.g. a CAR-T-cell) is infused to a recipient in need thereof. The infused cell is able to kill diseased cells expressing the target of the CAR in the recipient. Unlike antibody therapies, CAR-modified immune cells (e.g., CAR-T-cells) are able to replicate in vivo resulting in long-term persistence that can lead to sustained tumour control. In various embodiments, the CAR-T-cells are administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the CAR-T-cell to the patient.

The invention also includes a type of cellular therapy where immune cells (e.g., T-cells) are modified, e.g., by in vitro transcribed RNA, to transiently express a chimeric antigen receptor (CAR) and the CAR-T-cell is infused to a recipient in need thereof. The infused cell is able to kill tumour cells in the recipient. Thus, in various embodiments, the immune cells (e.g., CAR-T-cells) administered to the patient, is present for less than one month, e.g., three weeks, two weeks, one week, after administration of the CAR-T-cell to the patient. Without wishing to be bound by any particular theory, the anti-tumour immunity response elicited by the CAR-T-cells may be an active or a passive immune response, or alternatively may be due to a direct vs indirect immune response.

As noted above, ex vivo procedures are well known in the art and are described above. Briefly, cells are isolated from a mammal (e.g., a human) and genetically modified (i.e., transduced or transfected in vitro) with a vector expressing a CAR. The CAR-expressing cell (e.g., a CAR-T-cell) can be administered to a mammalian recipient to provide a therapeutic benefit. The mammalian recipient may be a human and the CAR-expressing cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, can be applied to the cells of the present invention. Other suitable methods are known in the art, therefore the present invention is not limited to any particular method of ex vivo expansion of the cells. Briefly, ex vivo culture and expansion of immune cells (e.g., T-cells) comprises: (1) collecting CD34+ hematopoietic stem and progenitor cells from a mammal from peripheral blood harvest or bone marrow explants: and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

The CAR-T-cells of the present invention may be administered either alone, or as a pharmaceutical composition in combination with diluents and/or with other components such as IL-2 or other cytokines or cell populations, as described herein. Immune cells may be administered either alone, or as a pharmaceutical composition in combination with diluents and/or with other components such as IL-2, IL-15, or other cytokines or cell populations. Briefly, pharmaceutical compositions may comprise immune cells as described herein, in combination with one or more pharmaceutically or physiologically acceptable carriers, diluents or excipients. Such compositions may comprise buffers such as neutral buffered saline, phosphate buffered saline and the like: carbohydrates such as glucose, mannose, sucrose or dextrans, mannitol: proteins: polypeptides or amino acids such as glycine: antioxidants: chelating agents such as EDTA or glutathione: adjuvants (e.g., aluminium hydroxide): and preservatives. Compositions for use in the disclosed methods are in some embodiments formulated for intravenous administration.

A pharmaceutical composition comprising the cells described herein may be administered at a dosage of 10⁴ to 10⁹ cells/kg body weight, such as 10⁵ to 10⁶ cells/kg body weight, including all integer values within those ranges. Cell compositions may also be administered multiple times at these dosages. The cells can be administered by using infusion techniques that are commonly known in immunotherapy (see, e.g., Rosenberg et al., 1988). The optimal dosage and treatment regime for a particular patient can readily be determined by one skilled in the art of medicine by monitoring the patient for signs of disease and adjusting the treatment accordingly.

In certain embodiments, it may be desired to administer activated immune cells to a subject and then subsequently re-draw blood (or have an apheresis performed), activate and expand the immune cells therefrom, and reinfuse the patient with these activated and expanded cells. This process can be carried out multiple times every few weeks. In certain embodiments, Immune cells can be activated from blood draws of from 10 cc to 400 cc. In certain embodiments, immune cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of immune cells.

### Combination Therapies

The immune cells, such as CAR-T-cells of the present invention, or produced by the methods of the present invention, may be co-formulated with and/or administered in combination with one or more additional therapeutically active component(s) selected from the group consisting of: a PRLR antagonist (e.g., an anti-PRLR antibody or small molecule inhibitor of PRLR), an EGFR antagonist (e.g., an anti-EGFR antibody [e.g., cetuximab or panitumumab] or small molecule inhibitor of EGFR [e.g., gefitinib or erlotinib]), an antagonist of another EGFR family member such as Her2/ErbB2, ErbB3 or ErbB4 (e.g., anti-ErbB2 [e.g., trastuzumab or T-DM1], anti-ErbB3 or anti-ErbB4 antibody or small molecule inhibitor of ErbB2, ErbB3 or ErbB4 activity), a cMET antagonist (e.g., an anti-cMET antibody), an IGFIR antagonist (e.g., an anti-IGFIR antibody), a B-raf inhibitor (e.g., vemurafenib, sorafenib, GDC-0879, PLX-4720), a PDGFR-alpha inhibitor (e.g., an anti-PDGFR-.alpha.antibody), a PDGFR-.beta. inhibitor (e.g., an anti-PDGFR-. beta. antibody or small molecule kinase inhibitor such as, e.g., imatinib mesylate or sunitinib malate), a PDGF ligand inhibitor (e.g., anti-PDGF-A, —B, —C, or -D antibody, aptamer, siRNA, etc.), a VEGF antagonist (e.g., a VEGF-Trap such as aflibercept, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354 such as REGN421), an Ang2 antagonist (e.g., an anti-Ang2 antibody disclosed in US 2011/0027286 such as HIH685P), a FOLH1 antagonist (e.g., an anti-FOLH1 antibody), a STEAP1 or STEAP2 antagonist (e.g., an anti-STEAP1 antibody or an anti-STEAP2 antibody), a TMPRSS2 antagonist (e.g., an anti-TMPRSS2 antibody), a MSLN antagonist (e.g., an anti-MSLN antibody), a CA9 antagonist (e.g., an anti-CA9 antibody), a uroplakin antagonist (e.g., an anti-uroplakin [e.g., anti-UPK3A] antibody), a MUC16 antagonist (e.g., an anti-MUC16 antibody), a Tn antigen antagonist (e.g., an anti-Tn antibody), a CLEC12A antagonist (e.g., an anti-CLEC12A antibody), a TNFRSF17 antagonist (e.g., an anti-TNFRSF17 antibody), a LGR5 antagonist (e.g., an anti-LGR5 antibody), a monovalent CD20 antagonist (e.g., a monovalent anti-CD20 antibody such as rituximab), a PD-1 antibody, a PD-L1 antibody, a CD3 antibody, a CTLA-4 antibody etc. Other agents that may be beneficially administered in combination with the CAR-T-cells of the invention include, e.g., tamoxifen, aromatase inhibitors, and cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors.

The immune cells, such as CAR-T-cells of the present invention, optionally produced by the methods of the present invention may be used in combination with a checkpoint inhibitor. In another example, an AKT inhibitor and/or an inhibitor of a PH domain protein can be administered with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L 1 (B7-H1: CD274) and PD-L2 (B7-DC: CD273) are expressed on the surface of APCs such as dendritic cells or macrophages. PD-L 1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L 1 (MDX-1105 (BMS-936559), MPDL3280A, MSB0010718C), PD-L2 (rHlgM12B7), CTLA-4 (Ipilimumab (MDX-010), Tremelimumab (CP-675,206)), IDO, B7-H3 (MGA271), B7-H4, TIM3, LAG-3 (BMS-986016).

In some embodiments, the PD-L 1 inhibitor comprises an antibody that specifically binds PD-L 1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L 1 antibodies and uses therefore are described in U.S. Pat. No. 8,552,154, which is incorporated by reference with respect to these antibodies. Anticancer agents comprising an anti-PD-1 antibody or anti-PD-L 1 antibody are described in U.S. Pat. No. 8,617,546, which is incorporated by reference with respect to these antibodies.

The present invention includes compositions and therapeutic formulations comprising any of the immune cells, such as CAR-T-cells, described herein in combination with one or more chemotherapeutic agents. Examples of chemotherapeutic agents include alkylating agents such as thiotepa and cyclosphosphamide (Cytoxan™): alkyl sulfonates such as busulfan, improsulfan and piposulfan: aziridines such as benzodopa, carboquone, meturedopa, and uredopa: ethylenimines and methylamelamines including altretamine, triethylenemelamine, trietylenephosphoramide, triethylenethiophosphaoramide and trimethylolomelamine: nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard: nitrosureas such as carmustine, chlorozotocin, fotemustine, lomustine, nimustine, ranimustine: antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin: anti-metabolites such as methotrexate and 5-fluorouracil (5-FU): folic acid analogues such as denopterin, methotrexate, pteropterin, trimetrexate: purine analogs such as fludarabine, 6-mercaptopurine, thiamiprine, thioguanine: pyrimidine analogs such as ancitabine, azacitidine, 6-azauridine, carmofur, cytarabine, dideoxyuridine, doxifluridine, enocitabine, floxuridine: androgens such as calusterone, dromostanolone propionate, epitiostanol, mepitiostane, testolactone: anti-adrenals such as aminoglutethimide, mitotane, trilostane: folic acid replenisher such as frolinic acid: aceglatone: aldophosphamide glycoside: aminolevulinic acid: amsacrine: bestrabucil: bisantrene: edatraxate: defofamine: demecolcine: diaziquone: elfornithine: elliptinium acetate: etoglucid: gallium nitrate: hydroxyurea: lentinan: lonidamine: mitoguazone: mitoxantrone: mopidamol: nitracrine: pentostatin: phenamet: pirarubicin: podophyllinic acid: 2-ethylhydrazide: procarbazine: PSK™: razoxane: sizofiran: spirogermanium: tenuazonic acid: triaziquone: 2,2′,2″-trichlorotriethylamine: urethan; vindesine: dacarbazine: mannomustine: mitobronitol: mitolactol: pipobroman: gacytosine: arabinoside (“Ara-C”): cyclophosphamide: thiotepa: taxanes, e.g. paclitaxel (Taxol™, Bristol-Myers Squibb Oncology, Princeton, N.J.) and docetaxel (Taxotere™: Aventis Antony, France): chlorambucil: gemcitabine: 6-thioguanine: mercaptopurine: methotrexate: platinum analogs such as cisplatin and carboplatin: vinblastine: platinum: etoposide (VP-16): ifosfamide: mitomycin C: mitoxantrone: vincristine: vinorelbine: navelbine: novantrone: teniposide: daunomycin: aminopterin: xeloda: ibandronate: CPT-11: topoisomerase inhibitor RFS 2000: difluoromethylornithine (DMFO); retinoic acid: esperamicins: capecitabine: and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston): and anti-androgens such as flutamide, nilutamide, bicalutamide, leuprolide, and goserelin: and pharmaceutically acceptable salts, acids or derivatives of any of the above.

The administration of any of the disclosed therapeutic agents may be carried out in any convenient manner, including by injection, transfusion, or implantation. The compositions described herein may be administered to a patient subcutaneously, intradermally, intratumourally, intranodally, intramedullary, intramuscularly, by intravenous (i.v.) injection, or intraperitoneally. In some embodiments, the disclosed compositions are administered by i.v. injection. The compositions may also be injected directly into a tumour, lymph node, or site of infection.

As will be appreciated by those skilled in the art, the above described cells will be administered to a subject in a therapeutically effective amount. The terms “effective amount” or “therapeutically effective amount” as used herein, refer to a sufficient amount of a therapeutic agent being administered which will relieve to some extent or prevent worsening of one or more of the symptoms of the disease or condition being treated. The result can be reduction or a prevention of progression of the signs, symptoms, or causes of a disease, or any other desired alteration of a biological system. For example, an “effective amount” for therapeutic uses is the amount of therapeutic agent required to provide a clinically significant decrease in disease symptoms without undue adverse side effects.

The term “therapeutically effective amount” includes, for example, a prophylactically effective amount. An “effective amount” of a therapeutic agent is an amount effective to achieve a desired pharmacologic effect or therapeutic improvement without undue adverse side effects. It is understood that “an effective amount” or “a therapeutically effective amount” can vary from subject to subject, due to variation in metabolism of the compound of any of age, weight, general condition of the subject, the condition being treated, the severity of the condition being treated, and the judgment of the prescribing physician.

It is considered well within the skill of the art for one to determine such therapeutically effective amounts by routine experimentation (including, but not limited to, a dose escalation clinical trial). An appropriate “effective amount” in any individual case may be determined using techniques, such as a dose escalation study.

Where more than one therapeutic agent is used in combination, a “therapeutically effective amount” of each therapeutic agent can refer to an amount of the therapeutic agent that would be therapeutically effective when used on its own, or may refer to a reduced amount that is therapeutically effective by virtue of its combination with one or more additional therapeutic agents.

### Methods of Treatment

The immune cells of or produced using the invention, e.g. CAR-T-cells, are useful, inter alia, for the treatment, prevention and/or amelioration of a disease or disorder. For example, the CAR-T-cells of the present invention are useful for the treatment of cancer, an infection, or an inflammatory disease. As another example, dendritic cells produced by a method of the invention can be used as a dendritic cell vaccine (see, for example, Datta et al., 2014) for treating, for example, a cancer, an infection (such as a bacterial or viral infection) or an autoimmune disease (such as diabetes). As a further example, NK cells, such as NK-CAR cells can be used to treat cancer (see, for example, Liu et al., 2021).

CAR-T-cells may be used to treat primary and/or metastatic tumours arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, CAR-T-cells of the invention are used to treat one or more of the following cancers: renal cell carcinoma, pancreatic carcinoma, head and neck cancer, prostate cancer, malignant gliomas, osteosarcoma, colorectal cancer, gastric cancer (e.g., gastric cancer with MET amplification), malignant mesothelioma, multiple myeloma, ovarian cancer, small cell lung cancer, non-small cell lung cancer, synovial sarcoma, thyroid cancer, breast cancer, melanoma, leukaemia, or lymphoma.

In an embodiment, the CAR-T-cells of the present invention are used to treat leukaemia, for example acute myeloid leukaemia, chronic myeloid leukaemia, acute lymphocytic leukaemia, or chronic lymphocytic leukaemia. In an embodiment, the leukaemia is acute myeloid leukaemia where low CD33+ blasts are dominant.

In another embodiment, the CAR-T-cells of the present invention are used to treat lymphoma, for example Hodgkin's lymphoma or non-Hodgkin's lymphoma. Non-Hodgkin's lymphoma types include diffuse large B-cell lymphoma, anaplastic large-cell lymphoma, Burkitt lymphoma, lymphoblastic lymphoma, mantle cell lymphoma, or peripheral T-cell lymphoma. In an embodiment, the lymphoma is diffuse large B cell lymphoma or non-Hodgkin's lymphoma with low levels of CD19 and/or CD20.

In the context of the methods of treatment described herein, the immune cells, such as CAR-T-cells, may be administered as a monotherapy (i.e., as the only therapeutic agent) or in combination (combination therapy) with one or more additional therapeutic agents (examples of which are described elsewhere herein).

In one embodiment, the subject is at risk of developing a cancer (e.g., cancer). A subject is at risk if he or she has a higher risk of developing a cancer than a control population. The control population may include one or more subjects selected at random from the general population (e.g., matched by age, gender, race and/or ethnicity) who have not suffered from or have a family history of a cancer. A subject can be considered at risk for a cancer if a “risk factor” associated with a cancer is found to be associated with that subject. A risk factor can include any activity, trait, event or property associated with a given disorder, for example, through statistical or epidemiological studies on a population of subjects. A subject can thus be classified as being at risk for a cancer even if studies identifying the underlying risk factors did not include the subject specifically.

In one embodiment, the subject is at risk of developing a cancer and the cells, or compositions, are administered before or after the onset of symptoms of a cancer. In one embodiment, the cells, or compositions are administered before the onset of symptoms of a cancer. In one embodiment, the cells, or compositions are administered after the onset of symptoms of a cancer. In one embodiment, the cells, or compositions of the present invention is administered at a dose that alleviates or reduces one or more of the symptoms of a cancer in a subject at risk.

In an embodiment, the subject has been diagnosed as having, or is suspected of having a disease or disorder such as cancer, infection or an inflammatory disease. In an embodiment, the subject has been diagnosed as having, or is suspected of having colon cancer or breast cancer. In an embodiment, the methods described herein comprise a step of diagnosing the subject as having or suspected of having disease or disorder such as cancer, infection or an inflammatory disease, preferably colon cancer or breast cancer.

Diagnosis as used herein refers to the determination that a subject or patient requires treatment with a CAR-T-cell and/or AKT inhibitor and/or an inhibitor of a PH domain protein of the invention. The type of disease or disorder diagnosed according to the methods described herein may be any type known in the art or described herein.

In an embodiment, the step of identifying or diagnosing a subject requiring treatment with a CAR-T-cell and/or AKT inhibitor and/or an inhibitor of a PH domain protein of the invention comprises the determination that the subject has cancer and may include assessment of one or more or all of:
blood profiling;
cell biopsy aspirates;
imaging such as computerized tomography (CT) scan, bone scan, magnetic resonance imaging (MRI), positron emission tomography (PET) scan, ultrasound and X-ray;
physical examination.

Examples of disease that can be treated with NK cells include, but are not limited to, cancers (e.g., melanoma, prostate cancer, breast cancer, and liver cancer) and infections, such as viral infections (e.g., infections by HSV, hepatitis viruses, human cytomegaloviruses, influenza viruses, flaviviruses, and HIV-1), bacterial infections (e.g., infections by Mycobacteria, Listeria, and Staphylococcus), and protozoan infections (e.g., infections by Plasmodium), and fungal infections (e.g., infections by Aspergillus).

As will be apparent to the skilled person a “reduction” in a symptom of a cancer in a subject will be comparative to another subject who also suffers from a cancer but who has not received treatment with a method described herein. This does not necessarily require a side-by-side comparison of two subjects. Rather population data can be relied upon. For example, a population of subjects suffering from a cancer who have not received treatment with a method described herein (optionally, a population of similar subjects to the treated subject, e.g., age, weight, race) are assessed and the mean values are compared to results of a subject or population of subjects treated with a method described herein.

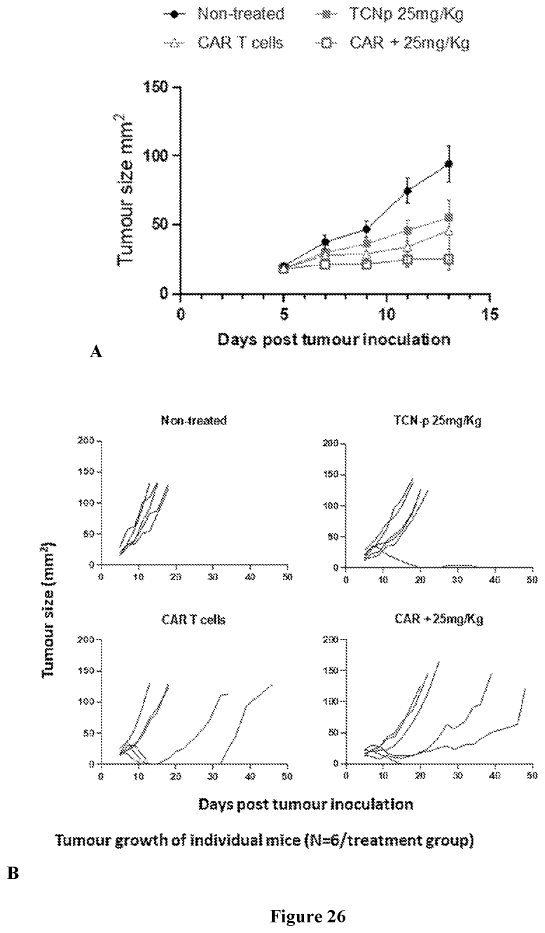

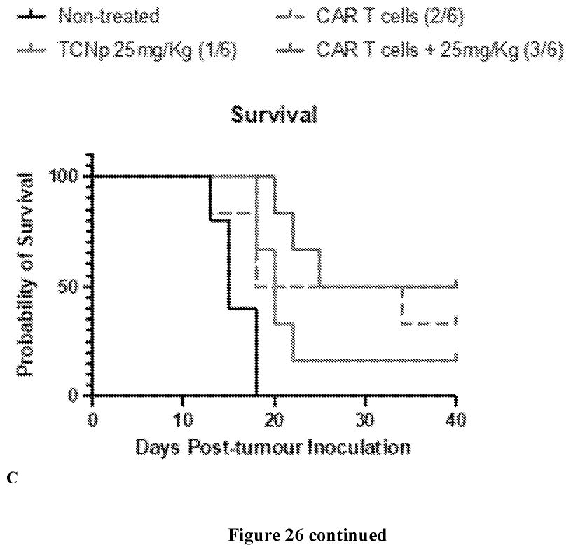

In one embodiment, the CAR-T-cells and methods of the present invention are used to improve survival of a subject suffering from a disease or disorder such as cancer, infection or an inflammatory disease. Where survival is contemplated, survival analysis can be performed using the Kaplan-Meier method (as shown in FIG. 26C). The Kaplan-Meier method estimates the survival function from life-time data and can be used to measure the fraction of patients living for a certain amount of time after treatment. A plot of the Kaplan-Meier method of the survival function is a series of horizontal steps of declining magnitude which, when a large enough sample is taken, approaches the true survival function for that population. The value of the survival function between successive distinct sampled observations (“clicks”) is assumed to be constant.

An important advantage of the Kaplan-Meier curve is that the method can take into account “censored” data-losses from the sample before the final outcome is observed (for instance, if a patient withdraws from a study). On the plot, small vertical tick-marks indicate losses, where patient data has been censored. When no truncation or censoring occurs, the Kaplan-Meier curve is equivalent to the empirical distribution.

In statistics, the log-rank test (also known as the Mantel-Cox test) is a hypothesis test to compare the survival distributions of two groups of patients. It is a nonparametric test and appropriate to use when the data are right censored. It is widely used in clinical trials to establish the efficacy of new drugs compared to a control group when the measurement is the time to event. The log-rank test statistic compares estimates of the hazard functions of the two groups at each observed event time. It is constructed by computing the observed and expected number of events in one of the groups at each observed event time and then adding these to obtain an overall summary across all time points where there is an event. The log-rank statistic can be derived as the score test for the Cox proportional hazards model comparing two groups. It is therefore asymptotically equivalent to the likelihood ratio test statistic based from that model.

### EXAMPLES

### Example 1—Materials and Methods

### Murine CAR-T Manufacturing Protocol

Mouse splenocytes were activated using CD3/CD28 antibodies and cultured in the presence of recombinant IL-2 and IL-7 for 24 hrs prior to transduction with CAR-T vectors. TCN (triciribine) or TCN-P (triciribine phosphate) were added immediately following transduction and the CAR-T-cells were then either exposed to TCN/TCN-P for 24, 48 or 72 hours.

Human PBMCs were extracted from buffy packs (Australian Red Cross Blood Service). They were activated using anti-CD3 antibody (OKT3) for 2 days before transduction for 48 hours, all in the presence of IL-2. TCN or TCN-P were added immediately to these CAR-T-cells cultured in IL-2 for up to 3 days.

### Tumour Killing Assay

100,000 E0771-Her2 tumour cells were seeded into each well of a 96-well plate and maintained at 37° ° C., 5% CO2. Two hours later, murine CAR-T-cells were seeded into the same wells at effector: target T-cell ratios of 2:1, 1:1 and 0.5:1) and incubated for up to 16 hours. The culture supernatants were collected and levels of interferon gamma (IFNγ) and tumour necrosis factor alpha (TNFα) were measured.

### Flow Cytometry Phenotyping

Cells were stained using fluorochrome labelled antibodies against CD4, CD8, CD44, CD62L to phenotype the murine CAR-T-cells following manufacturing. Human CAR-T-cells were phenotyped using fluorochrome-conjugated antibodies against CD4, CD8, CD45RA, CD45RO, CD44, CD62L, CCR7, CD27, PD-1 and CD69. A fixable live/dead dye was used to distinguish between live or dead cells.

### Mass Spectrometry

Human CAR-T-cells were collected after treatment with either TCN or TCN-P for either 24 or 3 days. At each time point, cells were collected and washed three times with cold DPBS before cell lysis for global proteomic analysis. Specifically, for phosphoproteomic analysis, human CAR-T-cells were either left as non-treated or treated with TCN or TCN-P for 0, 5 and 15 mins before washing with DPBS then cell lysis.

### Mass Spectrometry of Cellular Proteome and Phosphoproteome

Cells were homogenised/solubilised containing protease and phosphatase inhibitor (HALT) by tip-probe sonication, quantified, normalized and reduced (dithiothreitol, DTT), alkylated (iodoacetamide), before Sera-Mag Speed Bead-based protein digestion with LysC (enzyme:substrate 1:100, Wako Pure Chemical Industries) trypsin (enzyme:substrate 1:50). Tandem mass tag (TMT) multiplexing was performed for normalised peptide mixtures (9-plex TMT, reference 131C isobaric label). Peptides desalted (SDB-RPS Stage-Tips), and analysed for global cellular proteome. For phosphoproteome analysis, phosphopeptides were enriched from each TMT set using high select titanium dioxide (TiO2) bead capture.

Spectra acquired in data dependent acquisition on an Orbitrap Q-Exactive HF-X mass spectrometer coupled to an Easy-nLC 1200 (Thermo Fisher Scientific) ultra-high pressure liquid chromatography (UHPLC) pump. Peptides were separated (1.9-μm particle size C18, 0.075×250 mm, Nikkyo Technos Co. Ltd) with a gradient 5-100% buffer B (80% ACN, 0.1% FA) over 240 min at a flow rate of 300 nL/min with direct infusion at 55° C. Scan sequence included MS1 spectra (resolution of 60,000; mass range 300-1650 m/z: automatic gain control (AGC) target 3e6, max injection time of 128 ms, isolation window of 0.8 Th). The most intense MS1 ions were selected for MS2 analysis, fragmented by higher-energy collisional dissociation with normalized collision energy of 33. Precursors were filtered according to charge state≥2, AGC set at 1e5 for MS/MS, with monoisotopic peak used.

### Data Processing and Bioinformatics Pipeline

Mass spectra were pre-processed and processed using MaxQuant (1.6.14). Spectra searched against the full set of human protein sequences annotated in UniProt (sequence database January 2021) using Andromeda. Data was searched with fixed modification, cysteine carbamidomethylation and variable modifications, N-acetylation and methionine oxidation (and phosphorylation (STY)). Searches were performed using a 20 ppm precursor ion tolerance for total protein/phosphoprotein level analysis, and internal reference label channel to normalise batch-effect. Further modifications included TMT tags on peptide N termini/lysine residues (+229.16293 Da) set as static modifications. A stringent 1% false-discovery rate was applied to filter poor identifications at peptide and protein level. Resulting p values were adjusted by the Benjamini-Hochberg multi-test adjustment method for a high number of comparisons.

For further data analysis, the normalized intensities were converted into log 2 ratios of the intensities over the median intensity measured for each protein across each sample group, with statistical analyses performed using Student's T-test or ANOVA (p-value<0.05 was considered significant). Data analysis using Microsoft Office Excel, R (ggPlot2), and Perseus (Max-Planck Institute of Biochemistry, Department of Proteomics and Signal Transduction, Munich) software. Gene enrichment functional annotation clustering analysis was performed using Gprofiler/Reactome bioinformatics. All data were normalised to the internal reference TMT channel, and comparisons made either relative to DMSO and non-treatment controls (proteome analysis) and DMSO control at time 0 (phosphoproteome analysis). PH-containing domains were retrieved from SMART online software tool (http://smart.embl-heidelberg.de/).

### Example 2—Results

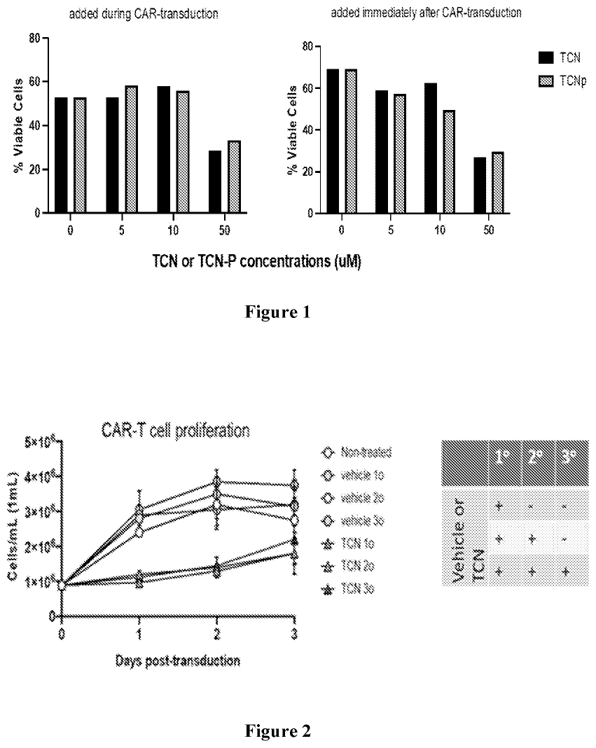

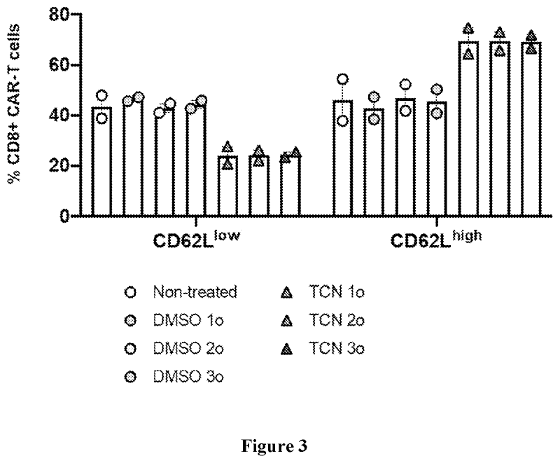

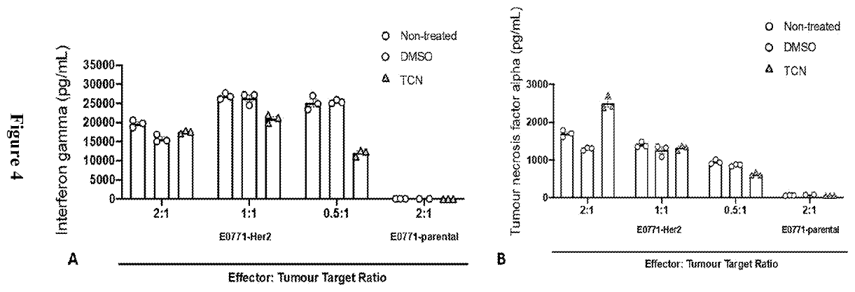

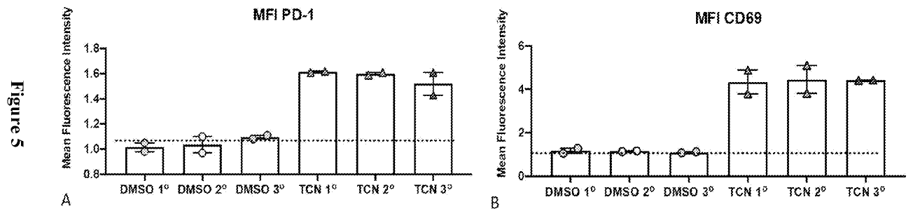

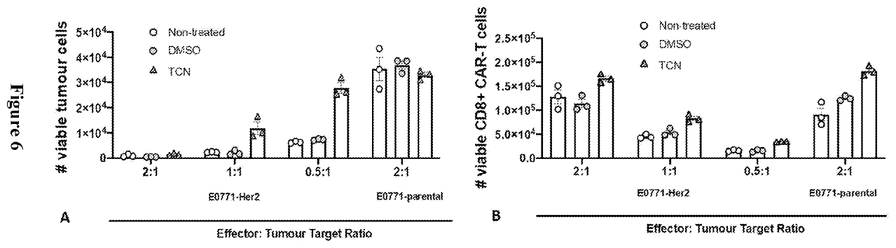

Adding 50 μM of TCN or TCN-P either during the process of transduction or afterwards resulted in at least 80% of CAR-T-cell death (FIG. 1). However, low concentrations of TCN or TCN-P (up to 10 μM, below 50 μM) maintained high levels of viable CAR-T-cells (˜60%), even up to 3 days of treatment. Proliferation rates were lower in murine CAR-T pre-treated with TCN (FIG. 2) while retaining a central memory phenotype (CD44^{hi}CD62L^{hi}) (FIG. 3) while retaining cytotoxic ability albeit producing slightly reduced levels of IFNγ and TNFα (FIGS. 4A-B).

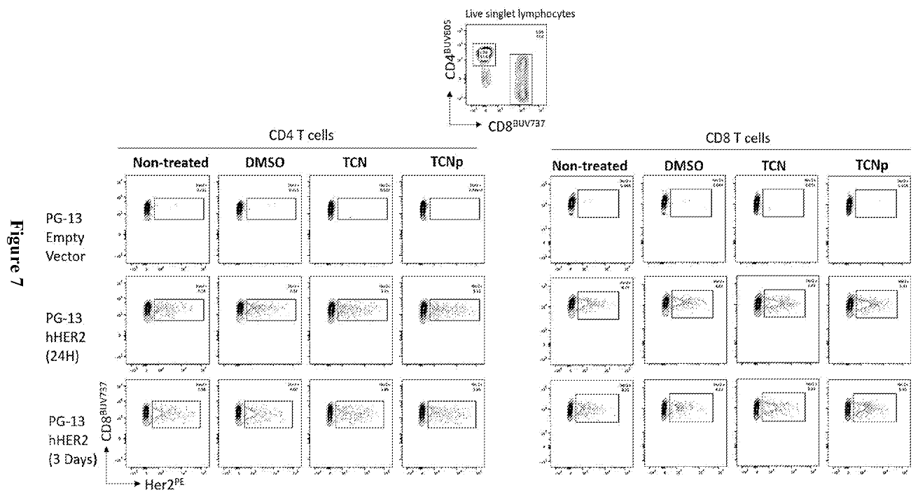

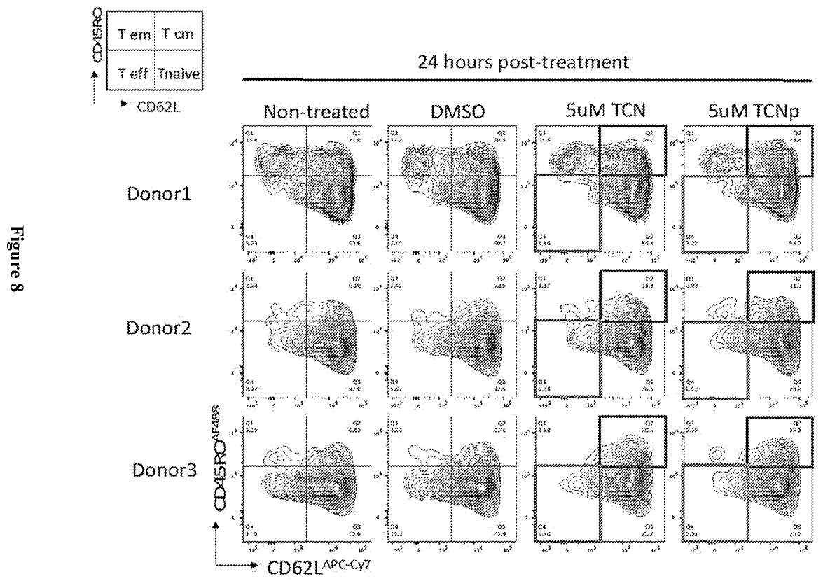

When activation markers were measured in murine CAR-T-cells, the inventors observed that TCN pre-treatment increased the expression of classical early activation markers PD-1 and CD69 (FIG. 5A-B). Tumour antigen directed cytotoxicity is retained following TCN pre-treatment, albeit blunted (FIG. 6A). In contrast, the survival of the murine CD8+ CAR-T-cells was slightly improved with TCN pre-treatment (FIG. 6B).

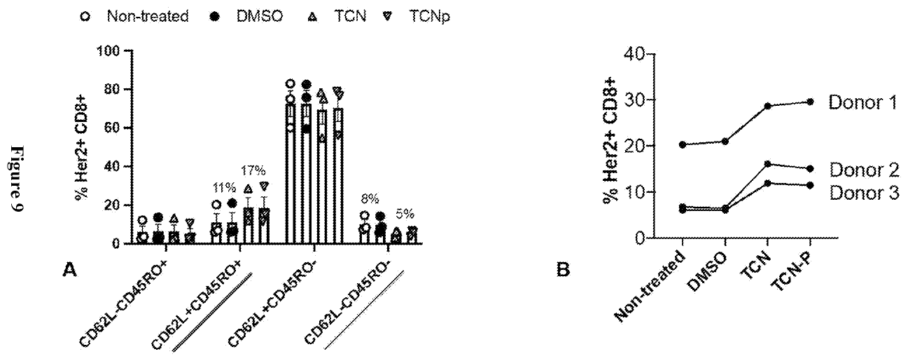

Based on these outcomes in murine CAR-T-cells, the same protocol was used to generate CAR-T-cells from human PBMCs. Buffy coats from three donors were processed to isolate PBMCs, where 50 million PBMCs were cultured for 2 days in the presence of media containing anti-CD3 antibodies (OKT3, 1.5 μg per mL) and recombinant IL-2 (600 U/mL). The activated T-cells were then subjected to a 2-day retroviral transduction protocol with a Her2-targeting CAR construct before treatment with 5 μM TCN or vehicle (DMSO) in the presence of 600 U/mL recombinant IL-2. Samples were collected either 24 or 72 hours later. Here, it was observed that TCN treatment did not influence transduction efficiency of CD4+ or CD8+ T-cells (FIG. 7).

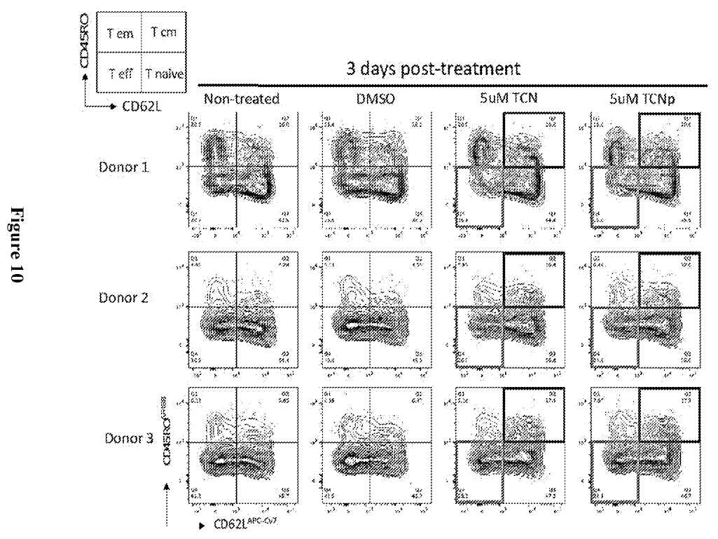

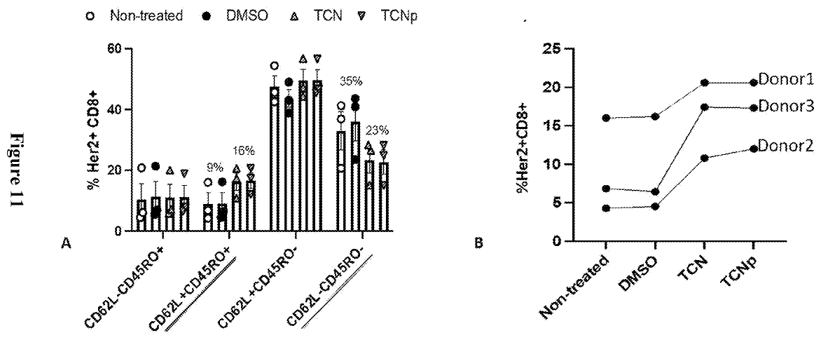

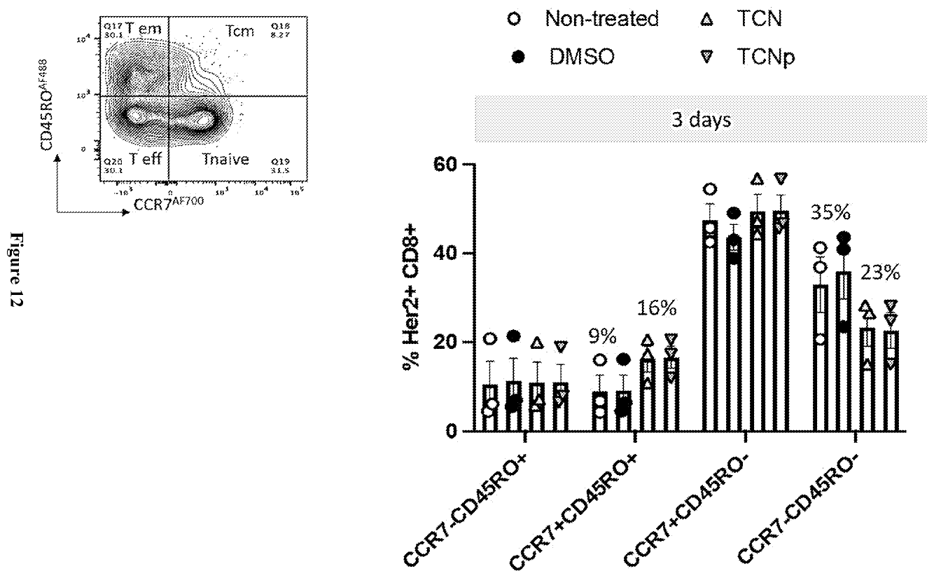

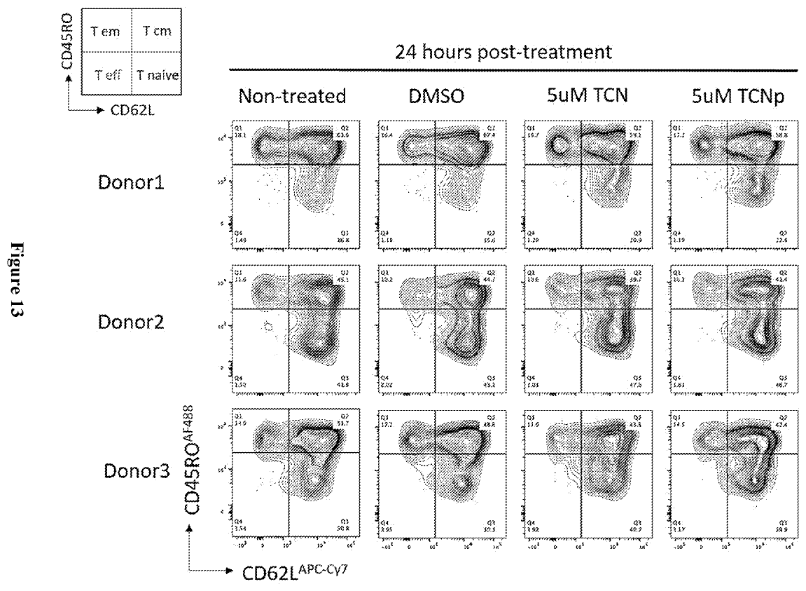

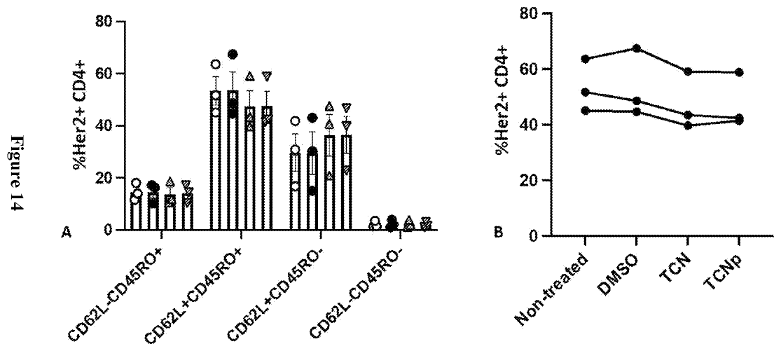

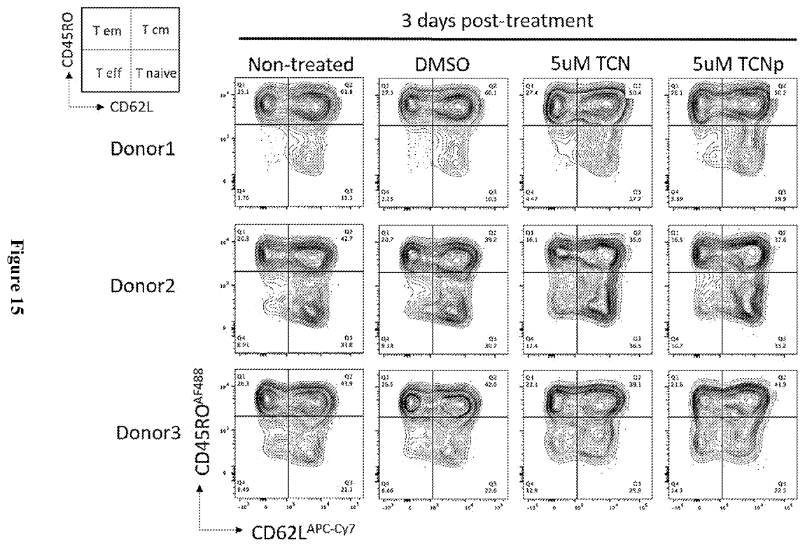

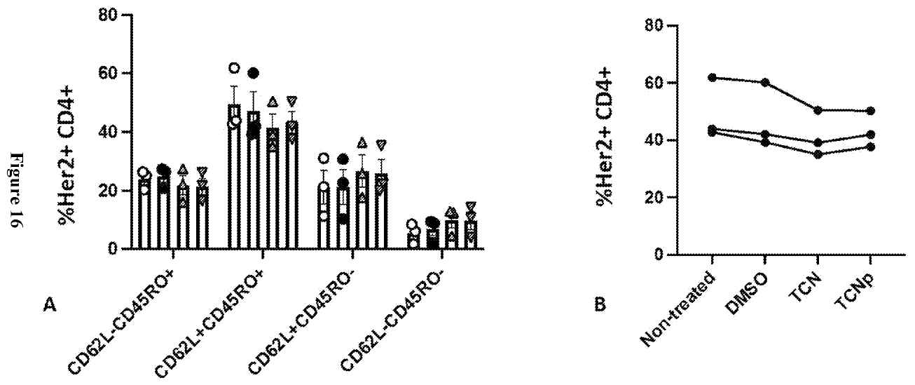

Interestingly, TCN treatment increased the proportion of central memory T (T_{CM}) cells consistently across all 3 donors (FIG. 8). When quantified across the three PBMC donors, it was noted that this increased in T_{CM} was concomitant (FIG. 9, 11% vs 17%) with a reduction in effector T-cells (T_{E}) (8% vs 5%). FIG. 9A shows the quantified data where data points a representative of each biological donor (i.e. individual PBMC donors) and FIG. 9B shows the changes to T_{CM} CAR-T-cells in response to vehicle (DMSO), TCN or TCN-P. It was also noted that this short exposure of CAR-T-cells to TCN or TCN-P sustains this shift towards T_{CM} cells for at least 3 days (FIG. 10-11, 9% vs 16%), concomitant with a reduction in effector T (T_{E}) cells (35% vs 23%). To avoid surface marker phenomenon, additional markers of T_{CM} cells were included (i.e. CD45RO and CCR7) where a similar pattern was observed with increased T_{CM} in CD8+ CAR-T following pre-treatment with TCN or TCN-P (FIG. 12). Interestingly, pre-treatment with TCN or TCN-P did not have any effect on the T_{CM} or T_{E} phenotype of CD4+ CAR-T-cells (FIGS. 13-16). This may have been due to the relatively high starting abundance of T_{CM} CD4+ CAR-T-cells (45-60% CD62L+CD45RO+ CD4+ CAR-T-cells, FIG. 14A).

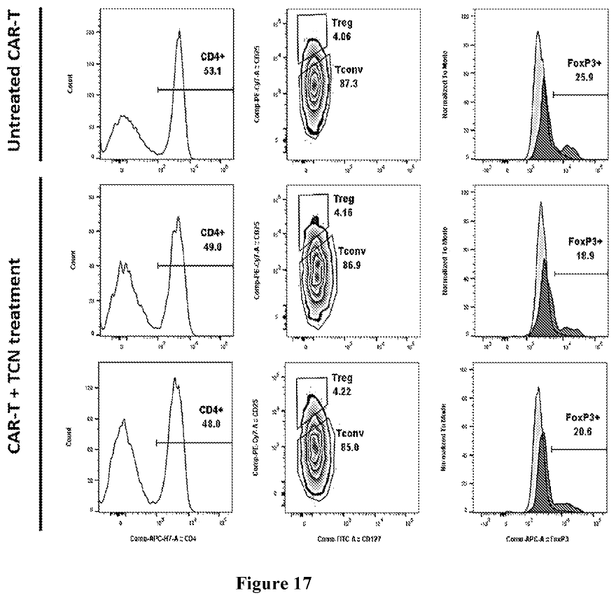

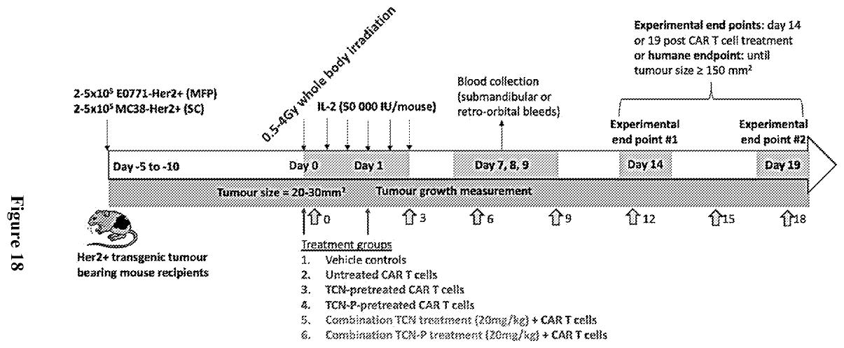

Given the role of regulatory T (T_{REG}) cells in the immunotolerance of the tumour microenvironment, the relative proportions of T_{REG} cells in the transduced human CAR-T-cells were also assessed. Here, it was observed that TCN pre-treatment reduced the T_{REG} proportions in CD4+ transduced CAR-T-cells (FIG. 17). TCN or TCN-P treatment of PBMCs increased enrichment of proteins associated with interferon signalling and anti-viral activity. These proteins included MX1-interferon-induced GTP-binding protein Mx1, Guanylate-binding protein-1 and -2, and DnaJ homology subfamily B member 1. Anti-viral activity is also suggested by the upregulation of LRRC59 which is crucial for nucleic acid sensing and TLR-3, 8 and -9 signalling. TCN or TCN-P treatment of PBMCs enriched for proteins associated with metabolism and RNA processing.

Significant dephosphorylation of multiple PH domain containing proteins as well as non-PH domain containing proteins where TCN or TCN-P are likely to be working upstream of them. Serine/threonine-protein kinase PRP4 homolog, spectrin beta chain, non-erythrocytic 1 which are important for regulatory T-cell function through the inhibition of TGFβ signalling. The data indicated that the following proteins are significantly inhibited following TCN administration: Oxysterol-binding protein 1, Oxysterol-binding protein 2, Spectrin beta chain, non-erythrocytic 1 and Rho GTPase-activating protein 27.

### Conclusions

Pre-treatment with TCN or TCN-P enabled enrichment in T-cell phenotypes which have been shown to persist in vivo and correspond with clinical response following CAR-T therapy. Given previous reports of toxicity to T-cells when used as an AKT inhibitor (see, e.g. Mousset et al., 2018), the findings of the current study were surprising. Extremely low concentrations of TCN and TCN-P were both well-tolerated by transduced T-cells, and efficacious in enriching for T-cells with a T_{CM} phenotype, including CCR7+CD45RO+CD8+ T-cells. Furthermore, pre-treatment with low concentrations of TCN or TCN-P reduced T_{REG} cells in transduced CAR-T-cells.

Together, the findings demonstrate that TCN or TCN-P pre-treatment is an efficacious method for enhancing the efficacy of CAR-T therapies by enriching for T-cell phenotypes that are known to persist in vivo and are associated with partial or complete clinical response. TCN and TCN-P pre-treatment appear to largely affect the CD8+ CAR-T-cells, with little or no effects on CD4+ CAR-T-cells.

The data further indicated that the inhibitor can be used to increase dendritic cell numbers and the cytotoxic activity of NK cells.

### Example 3—Materials and Methods for Testing Efficacy of TCN and TCN-P as Pre-Treatment or as a Neoadjuvant In Vivo

The inventors next sought to determine the effects of AKT inhibitor on the enhancement of T_{CM} and CAR-T_{SCM} phenotypes in vivo in therapeutically relevant models of cancer. The first approach was to test the effect of an AKT inhibitor when used as a manufacturing reagent to enrich CAR-T_{CM} and CAR-T_{SCM} phenotypes in either endogenous or adoptively transferred CAR-T, as per the schematic illustrated in FIG. 18. The second approach was to test the effect of an AKT inhibitor when used as an adjuvant to enrich CAR-T_{CM} and CAR-T_{SCM} phenotypes in either endogenous or adoptively transferred CAR-T, as per the schematic illustrated in FIG. 24.

### Cell Lines and Mice

The mouse colon adenocarcinoma MC-38-hHer2 and breast carcinoma E0771-hHer2 cancer cell lines were used in in vitro and in vivo experiments. The GP+e86 cell line used to produce retroviral vectors were obtained from ATCC (VA, USA). All tumour and packaging cell lines were maintained at 37° C. with 5% CO₂ in RPMI medium supplemented with 10% heat-inactivated fetal bovine serum, 2 mM glutamine, 1 mM sodium pyruvate, 0.1 mM non-essential amino acids, 10 mM HEPES, 100 U mL-1 penicillin and 100 ug mL⁻¹ streptomycin. All cell lines were tested mycoplasma negative.

C57BL/6J wildtype (WT) and human Her2 (hHer2) transgenic mouse lines were all in bred and maintained at the Peter MacCallum Cancer Centre (Victoria, Australia). Mice between the age of 6 and 16 weeks were gender-matched and randomised into different treatment groups. All animal experiments were approved by the Animal Experimental Ethics Committee (Protocol E678).

### Retroviral Transduction of Murine CAR-T-Cells

Splenocytes from C57BL/6 donor mice were activated with 1 μg mL⁻¹ anti-CD3, 1 μg mL-1 anti-CD28 in the presence of IL-2 and 200 pg mL-1 IL-7 for 1 day then ficoll gradient treatment. Cells were transduced in plates coated with 1 μg mL of retronectin using supernatant produced from a GP+86 LXSN-anti-Her2 CAR packaging cell line. The transduced cells were then expanded in complete RPMI with the same concentration of IL-2 and IL-7 with or without 5 μM of PTX-200. Fresh IL-2 and IL-7 in complete media were added to all CAR-T-cells 2 days post-transduction.

### Human Her2+ Syngeneic Mouse Tumour Model.

Her2 transgenic recipients were either injected subcutaneously with 2.5×10⁵ of MC38-hHer2 or 2.5×10⁵ of E0771-hHer2 into the mammary fat pad with. Once the tumours become palpable and they were measured using a manual caliper and tumour area calculated in millimetres squared (mm², length×width). On day 6-7 post-tumour inoculation, tumour-bearing Her2+ mice were ranodmised to have an average tumour size of 20 mm², 20×10⁶ of CAR-transduced T-cells were adoptively transferred into these recipients. 50 000 U of IL-2 was administered via intraperitoneal injection 5 times over the next 2 days. Tumour sizes were measured and monitored every 2-3 days until their tumour size exceed 120 mm² or until the tumour-bearing mice reached a designated experimental time point.

### Intraperitoneal Injection of PTX-200

PTX-200 diluted in DPBS was administered at 5, 25 or 50 mg/Kg per mouse every 3-4 days via intraperitoneal injection.

### Analysis of Immune Subsets at the Tumour Site, Draining Lymph Node, Spleen and Blood of Mice

Blood of mice was collected directly into EDTA-containing tube via submandibular bleeding at day 8-9 post-treatment. At the experimental endpoint, various organs including tumour, draining lymph node (dLN) and spleen were collected immediately after euthanasia. Single cell suspension from the dLN and spleen were achieved by processing these organs through a 70 μm filter. For splenocytes, red blood cells were lysed with ACK lysis buffer. Solid tumours were manually sliced and digested with DMEM containing 1 mg mL⁻¹ type IV collagenase and 0.02 mg mL⁻¹ DNAse for 30 mins at 37° C. in a rocking incubation at 120 r.p.m. Digestion was neutralised with DMEM and processed through a 70 μm filter and resuspended in either FACS buffer or complete RPMI media. To restimulate cells ex vivo, single cell suspension from these different tissues were resuspended in supplemented RPMI media and activated with phorbol 12-myristate 12-acetate (PMA: 10 ng mL⁻¹), calcium ionophore (1 μg mL⁻¹) with the addition of Golgi Plug (BD Biosciences) and STOP (BD Biosciences) for 4 hours at 37° C. before analysis of flow cytometric analysis.

### In Vitro Chronic Tumour Restimulation

CAR-T-cells were co-cultured with either MC-38 or E0771-hHer2 cancer cells at the indicated Effector: Target ratio in the presence of IL-7 (200 pgmL⁻¹) and IL-15 (10 ngmL⁻¹). After 1 day, CAR-T-cell suspension was collected and co-cultured with a fresh layer of cancer cells. This was repeated three times with IL-7 and IL-15. Supernatants were collected every day and analysed with Cytokine Bead Array while cells after the 3rd round of tumour restimulation were subjected to flow cytometric analysis.

### Analysis of Live Cancer Cells Over Time

Non-treated or PTX-200 pre-treated CAR-T-cells were co-cultured with either MC-38 or E0771-hHer2 at the indicated Effector: Target ratio in a 384 well plate. Caspase 3/7 dye were dispensed into cell suspension. Images were taken every 4 hours using the Incucyte SX5. Cell count and associated Caspase 3/7 activity were quantified using the IncuCyte Zoom software.

### Flow Cytometry

Cells were blocked for 15 mins at room temperature with Fc receptor block (clone 2.4G2 of the hybridoma supernatant diluted 1:50 with FACS buffer). Cells were washed with FACS buffer and then stained with fluorochrome-conjugated antibodies for 30 mins on ice. Stained cells were washed twice with FACS buffer before being resuspended in FACS buffer with 20 000 counting beads. For intracellular or intranuclear stains, stained cells were then fixed and permeabilised with either the BD Biosciences or eBioscience kit as per manufacturer instructions, respectively. Fixed and permeabilised cells were stained with flurochrome-conjugated antibodies for 30 mins at room temperature before washing with perm/wash buffer twice and final resuspension in FACS buffer with counting beads. Stained samples were acquired on BD FACSymphony (BD Biosciences). The number of target T-cell population was calculated by using: the number of input beads in the sample/bead events×events of targeted population.

### Statistical Analysis

FACS analysis were performed using Flowjo vs. 10. Figures and statistical analysis were generated using version 9 GraphPad PRISM software. Data are represented as mean±s.e.m. and statistical analysis were performed using one-way/two-way analysis of variance (ANOVA) while the log-rank (two-tailed Mantel-Cox) test was used for determining the statistical significance in survival analysis.

### Example 4—Results of the Efficacy of TCN and TCN-P as a Pretreatment In Vivo

The inventors first sought to determine the effect of an AKT inhibitor when used as a manufacturing reagent to enrich CAR-T_{CM} and CAR-T_{SCM} phenotypes in either endogenous or adoptively transferred CAR-T, as per the schematic illustrated in FIG. 18.

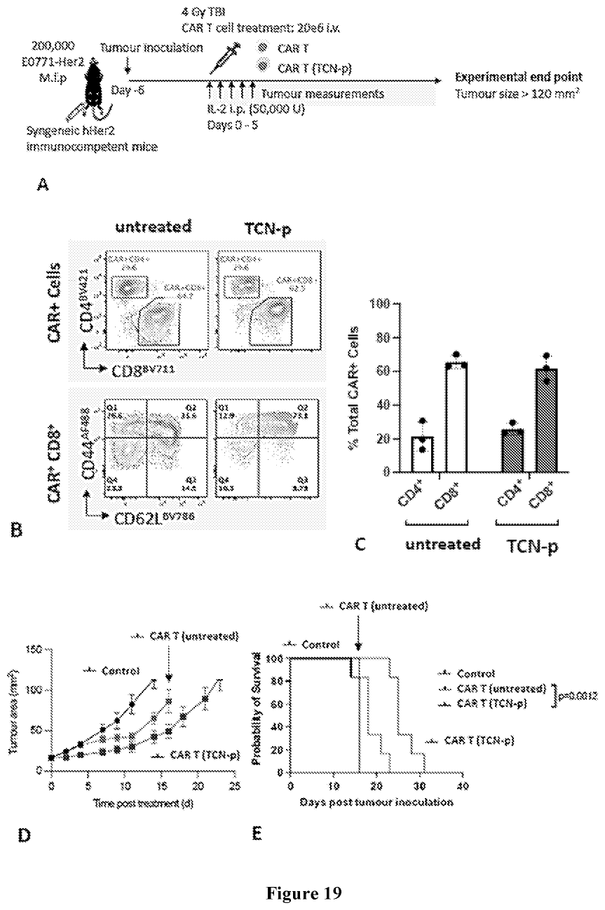

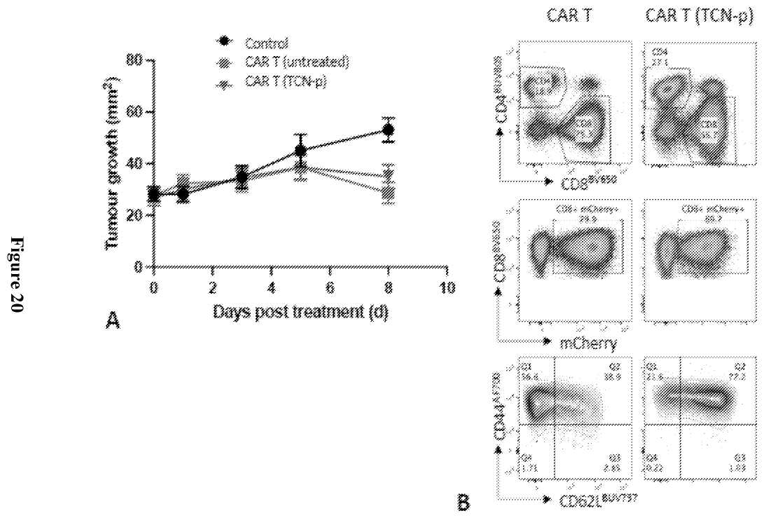

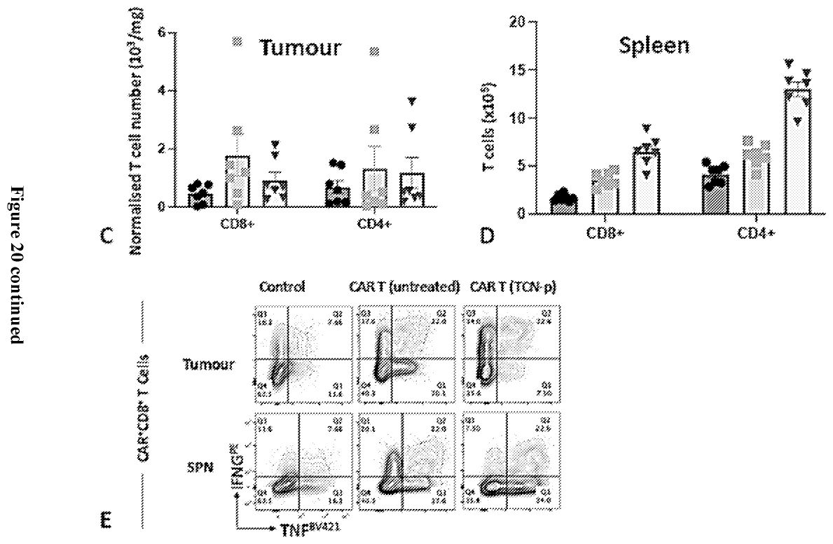

A representative example of the tumour model in immunocompetent syngeneic hHer2 mice is shown in FIG. 19A. Prior to administration of CAR-T-cells, flow cytometry is employed to determine the ratio of CD4:CD8 T-cells where it was observed that this unchanged by TCN-P conditioning during CAR-T manufacturing (FIG. 19B, upper panel and FIG. 19C). However, the TCN-P preconditioning conferred a significant enrichment in central memory CD8+ CAR-T-cells (FIG. 19B, lower panel). The impact of TCN-P preconditioning on CAR-T-cells translated to improved tumour control in vivo. When left uncontrolled, the E0771 breast cancer tumour model reached experimental endpoints by day 14 (i.e. tumour>120 mm²). CAR-T administration was able extend this until day 16, and pre-conditioned CAR-T-cells were able to extend this until day 24 (FIG. 19D), and a significant improvement in probability of survival (FIG. 19E, p=0.0012).

Using the same syngeneic hHer2 breast cancer model, a separate cohort of animals were evaluated for tumour growth until 8 days post-treatment (FIG. 20A). Analysis of circulating T-cells showed that preconditioning of CAR-T-cells resulted in skewing of the CD4:CD8 ratio in vivo such that CD4+ CAR-T-cells were more prevalent in the animals that received TCN-P preconditioned CAR-T-cells compared to those that received untreated CAR-T-cells (FIG. 20B, upper panel). It was also noted that preconditioning resulted in fewer CD8+ CAR-T-cells in vivo (FIG. 20B, middle panel). These observations coincided with near doubling of the central memory T-cells (FIG. 20B, bottom panel).

Flow cytometric analysis further revealed that preconditioning of CAR-T-cells did not have significant impact on CD4+ or CD8+ compositions within the tumour (FIG. 20C) but appeared to increase CD4+ cells within the spleen—a secondary lymphoid organ (FIG. 20D). Interestingly, the improvements in tumour killing efficiency by preconditioned CAR-T-cells did not coincide with any significant changes to production of cytokines, TNF-alpha or IFN-gamma (FIG. 20E).

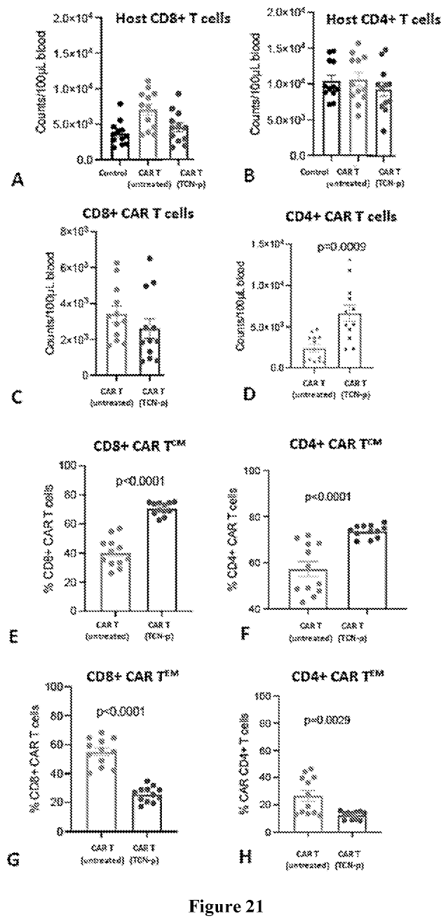

Circulating numbers of host CD4+ and CD8+ T-cells were not significantly between groups (FIG. 21A-B). CD8+ CAR-T-cells were also similar between groups but circulating CD4+ CAR-T-cells were increased by an average of 3-fold in the animals that received preconditioned CAR-T-cells (FIG. 21D, p=0.0009). Phenotyping of circulating CAR-T-cells suggested that the preconditioning during the manufacturing step enabled CAR-T-cells to retain the central memory phenotype in vivo. The percentage of CD8+ CAR-T-cells with central memory phenotype was nearly double in the preconditioned group (FIG. 21E, p<0.0001). The percentage of CD4+ CAR-T-cells with central memory phenotype was about 20% higher in the preconditioned group (FIG. 21F, p<0.0001). These findings coincided with the halving of CD8+ and CD4+ CAR T-cells with effector memory phenotype (FIG. 21G-H, p<0.001 and p=0.0029 respectively).

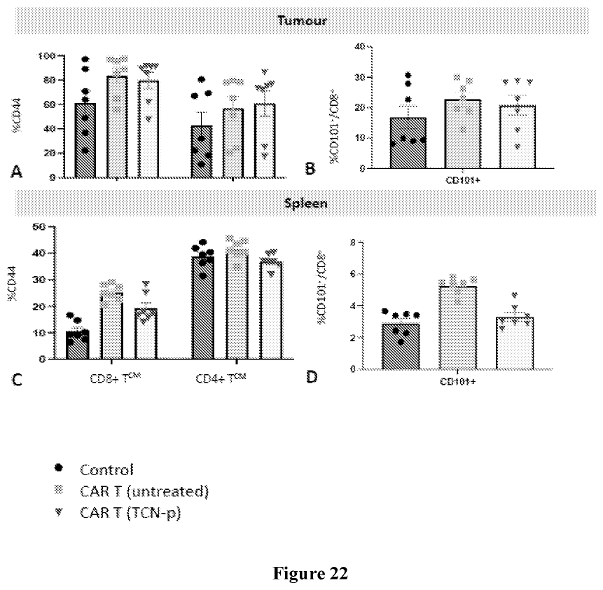

When the immune cells of the tumour and spleens were analysed, we observed that preconditioning had no significant effect on the CD8+ or CD4+ CAR-T-cell bearing the central memory phenotype (FIGS. 22A & C). While no significant differences were observed in the expression of early exhaustion marker on the CD8+ CAR-T-cells in the tumours (FIG. 22B), preconditioning appeared to protect CD8+ CAR-T-cells against exhaustion when recruited to the spleen (FIG. 22D).

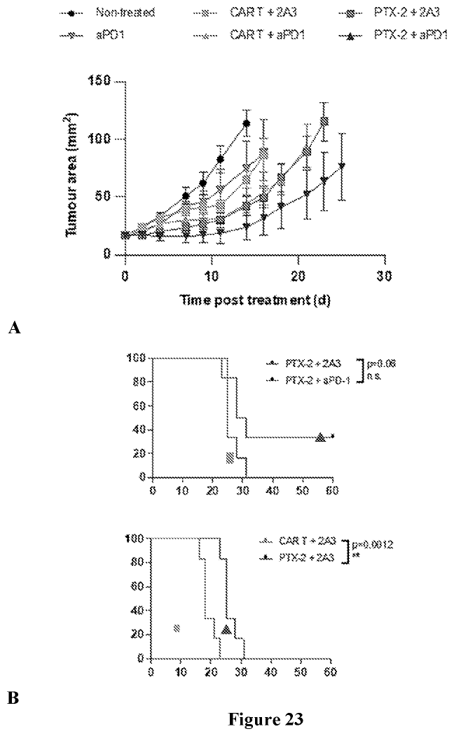

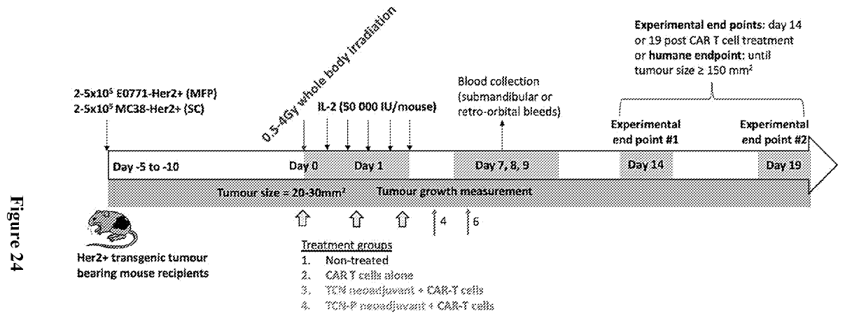

The inventors then sought to determine the effect of CAR-T-cells pre-treated with TCN-P in combination with PD-1 checkpoint blockade to determine whether this treatment improves anti-tumour efficacy against solid tumours. As shown in FIG. 23A-B, when CAR-T-cells were pre-treated with TCN-P in combination with PD-1 checkpoint blockade a synergistic reduction in tumour area was observed.

### Example 5—Results of the Efficacy of TCN and TCN-P as a Neoadjuvant In Vivo

In second approach, the effect of an AKT inhibitor when used as an adjuvant to enrich CAR-T_{CM} and CAR-T_{SCM} phenotypes in either endogenous or adoptively transferred CAR-T was tested, as per the schematic illustrated in FIG. 24.

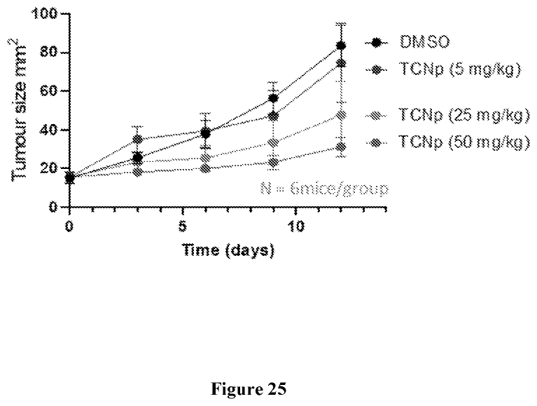

As shown in FIG. 25, when Her2+ transgenic mice were inoculated with 2.5€5 MC-38-hHer2 and subsequently intraperitoneally injected with either DMSO (vehicle control) or TCN-P at 5, 25 or 50 mg/Kg every 3-4 days, TCN-P induced a strong anti-tumour effect demonstrating its ability to exert an effect on tumour growth in vivo. The effect of TNC-p was then tested in conjunction with CAR-T-cell therapy. As shown in FIG. 26A-B, mice receiving the combinatory treatment of intravenous injection with 20×10⁶ anti-human Her2 CAR-T-cells and TCN-P at 25 mg/Kg demonstrated significantly reduced tumour size when compared to the single treatment alone. Consistently, mice receiving the combinatory treatment of intravenous injection with 20×10⁶ anti-human Her2 CAR-T-cells and TCN-P at 25 mg/Kg also demonstrated increased survival rates when compared to the single treatment alone (FIG. 26C). In particular, the median survival duration for the control group was 15 days and 20 days for mice treated with TNC-p at 25 mg/Kg. In the presence of CAR-T-cells, the survival duration increased to 26 days however in the presence of both the CAR-T-cells and TCN-P, survival increased to 32.5 days.

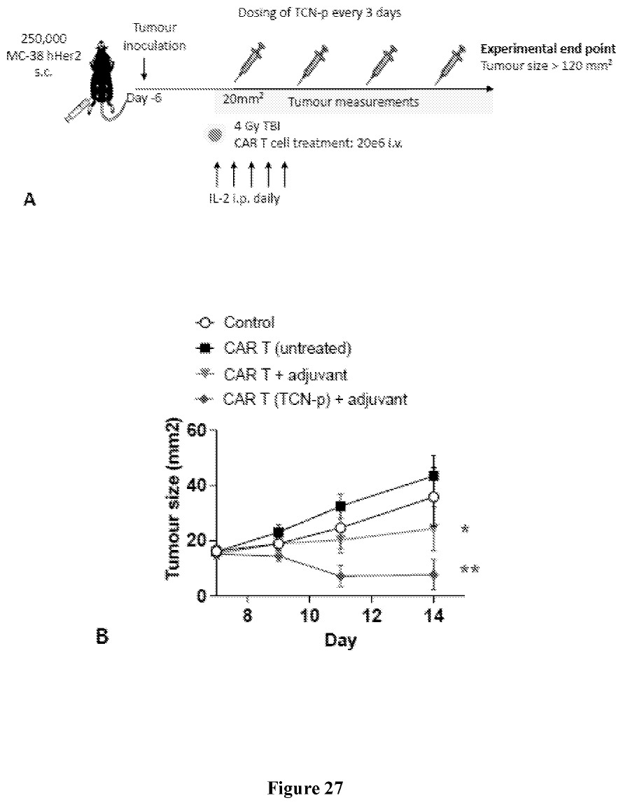

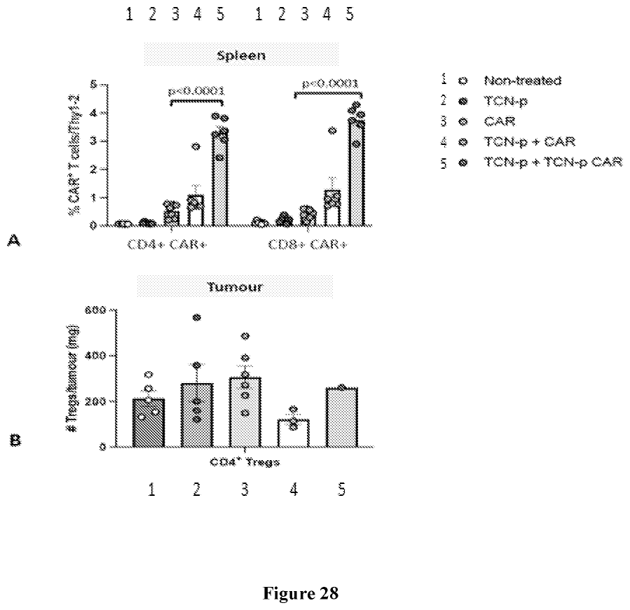

Finally, the utility of TCN-P as an adjuvant for CAR-T therapy was also assessed using the MC-38 hHer2 model of colon cancer where mice were either administered vehicle, untreated CAR-T-cells, CAR-T-cells in combination with 25 mg/kg TCN-P adjuvant every 3 days following CAR-T administration, or a combination of pretreated CAR-T-cells and TCN-P adjuvant (FIG. 27A). Tumour sizes until day 14 was assessed. Here the inventors observed that the administration of TCN-P as an adjuvant significantly improved tumour control by Her2-targeting CAR-T-cells (FIG. 27B, *p<0.05) where the combination of preconditioned CAR-T-cells and TCN-P adjuvant resulted in the most significant tumour killing in vivo (p<0.01). Unexpectedly, is was determined that the spleens contained a significant accumulation and/or expansion of CD4+ and CD8+ CAR T-cells (p<0.0001) in response to the combination of TCN-P pretreatment and TCN-P adjuvant therapy (FIG. 28A). Moreover, the administration of TCN-P as an adjuvant in the presence of CAR-T-cells resulted in a 50% reduction of intratumoral Tregs (FIG. 28B).

The findings from this study demonstrate that TCN-P preconditioning can enhance the efficacy of conventional CAR-T therapies by enriching for T-cell phenotypes that are known to persist in vivo and are associated with partial or complete clinical response. Given the clinical safety data around the use of TCN-P in oncology indications, it is plausible that TCN-P and similar compounds can be used in conjunction with a CAR-T therapy in order to elevate in vivo persistence of central memory CAR-T-cell, and in so doing, improve clinical durability and response.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Berg et al. (1998) Transplant Proc. 30:3975-3977.
Datta et al. (2014) Yale J Biol Med 87:491-518.
Garland et al. (1999) J. Immunol Meth. 227:53-63.
Ghosh et al. (1991) Glycobiology 5:505-510.
Haanen et al. (1999) J. Exp. Med. 190:1319-1328.
Lui et al. (2021) J Hemat Oncol 14:7.
McLellan and Ali Hosseini Rad (2019) Immunol Cell Biol 97:664-674.
Mousset et al. (2018) Oncoimmunology 7:e1488565.
Rosenberg et al. (1988) New Eng. J. of Med 319:1676.
Sabado et al. (2017) Cell Res 27:74-95.
Smith et al. (2015) Clinical & Translational Immunology (2015) 4:e31.

1. A method of modifying an immune response in a subject, the method comprising administering to the subject a population of immune cells, wherein the immune cells were produced using a method comprising culturing immune cells in medium comprising an AKT inhibitor and/or an inhibitor of a PH domain protein, preferably wherein the immune cells are T-cells, dendritic cells, natural killer cells, myeloid cells, macrophages or a combination thereof.

2. A method of modifying a T-cell response in a subject, the method comprising administering to the subject a population of T-cells comprising a chimeric antigen receptor (CAR-T-cells), wherein the CAR-T-cells were produced using a method comprising culturing CAR-T-cells in medium comprising an AKT inhibitor and/or an inhibitor of a PH domain protein.

3. A method of modifying an immune response in a subject, preferably a T-cell response, the method comprising:
a) administering to the subject an AKT inhibitor and/or an inhibitor of a PH domain protein, and
b) at least about 18 hours after step a) administering to the subject a population of immune cells, preferably comprising T-cells comprising a chimeric antigen receptor (CAR-T-cells).

4. A method of modifying a dendritic cell and/or natural killer cell response in a subject, the method comprising:
a) administering to the subject an AKT inhibitor and/or an inhibitor of a PH domain protein, and
b) administering to the subject a population of immune cells comprising dendritic cell and/or natural killer cells.

5. The method of claim 3 or claim 4, wherein the population of immune cells are administered between about 18 hours and about 72 hours after the AKT inhibitor and/or an inhibitor of a PH domain protein.

6. A method of reducing cytokine release syndrome (CRS) in a subject undergoing CAR-T-cell therapy, the method comprising administering to the subject an AKT inhibitor and/or an inhibitor of a PH domain protein and/or CAR-T-cells, wherein the CAR-T-cells administered to the subject have been cultured in a medium comprising the AKT inhibitor and/or an inhibitor of a PH domain protein.

7. The method of claim 6, wherein the chimeric antigen receptor comprises a CD28z co-stimulatory domain.

8. Use of an AKT inhibitor and/or an inhibitor of a PH domain protein for the manufacture of a medicament for modifying an immune response in a subject, preferably a T-cell response, wherein the subject will be administered with a population of immune cells, preferably comprising T-cells comprising a chimeric antigen receptor (CAR-T-cells), at least 18 hours after the medicament.

9. Use of a population of immune cells, preferably comprising T-cells comprising a chimeric antigen receptor (CAR-T-cells) for the manufacture of a medicament for modifying an immune response in a subject, preferably a T-cell response, wherein the subject will be or has been administered with an AKT inhibitor and/or an inhibitor of a PH domain protein at least 18 hours before the medicament.

10. Use of an AKT inhibitor and/or an inhibitor of a PH domain protein for the manufacture of a medicament for modifying an immune response in a subject, preferably a T-cell response, wherein the medicament is CAR-T-cells.

11. Use of an AKT inhibitor and/or an inhibitor of a PH domain protein for the manufacture of a medicament for modifying a dendritic cell and/or natural killer cell response in a subject.

12. Use of a population of immune cells comprising dendritic cells and/or natural killer cells for the manufacture of a medicament for modifying a dendritic cell and/or natural killer cell response in a subject, wherein the subject has been, or will be, administered with an AKT inhibitor and/or an inhibitor of a PH domain protein.

13. An AKT inhibitor and/or an inhibitor of a PH domain protein for use in producing a population of immune cells, preferably comprising T-cells comprising a chimeric antigen receptor (CAR-T-cells), for modifying an immune response in a subject, preferably a T-cell response in a subject.

14. An AKT inhibitor and/or an inhibitor of a PH domain protein for use in modifying an immune response in a subject, preferably a T-cell response, wherein the subject will be administered with a population of immune cells, preferably comprising T-cells comprising a chimeric antigen receptor (CAR-T-cells), at least 18 hours after the medicament.

15. A population of immune cells comprising T-cells comprising a chimeric antigen receptor (CAR-T-cells) for use in modifying an immune response in a subject, preferably a T-cell response, wherein the subject will be or has been administered with an AKT inhibitor and/or an inhibitor of a PH domain protein at least 18 hours before the medicament.

16. A method for modifying an immune response in a subject, the method comprising administering to the subject a population of immune cells and a checkpoint inhibitor, wherein the immune cells were produced using a method comprising culturing immune cells in medium comprising an AKT inhibitor and/or an inhibitor of a PH domain protein, preferably wherein the immune cells are T-cells, dendritic cells, natural killer cells, myeloid cells, macrophages or a combination thereof.

17. The method of claim 16, wherein the immune cells are T-cells comprising a chimeric antigen receptor (CAR-T-cells) and the checkpoint inhibitor is an anti-PD-1 antibody.

18. The method of claim 17, wherein the treatment is synergistic when compared to the effect of the either treatment alone.

19. A method for modifying an immune response in a subject, preferably a T-cell immune response, the method comprising administering to the subject:
(i) a population of immune cells, preferably produced using a method comprising culturing immune cells in medium comprising an AKT inhibitor and/or an inhibitor of a PH domain protein, wherein the immune cells are T-cells, dendritic cells, natural killer cells, or a combination thereof, most preferably T-cells comprising a chimeric antigen receptor (CAR-T-cells); and
(ii) an AKT inhibitor and/or an inhibitor of a PH domain protein.

20. The method of any one of claims 1 to 7 or 16 to 19, the use of any one of claims 8 to 12, the AKT inhibitor and/or an inhibitor of a PH domain protein for use according to claim 13 or 14 or the population of immune cells according to claim 15, wherein modification of the immune response or T-cell immune response increases survival of the subject when compared to a subject not receiving CAR-T-cells and/or an AKT inhibitor and/or an inhibitor of a PH domain protein.

21. The method, the use, AKT inhibitor and/or an inhibitor of a PH domain protein for use or the population of immune cells of claim 20, the wherein survival is increased by 3, 6, 9, 12, 24, 36, 48, 60, 72, 84, 96 months or more.

22. The method of any one of claims 1 to 7 or 16 to 21, the use according to any one of claims 8 to 12 or 20 to 21, the AKT inhibitor and/or an inhibitor of a PH domain protein for use according to any one of claims 13 to 14 or 20 to 21 or the population of immune cells according to any one of claims 15 or 20 to 21, wherein the subject is immunodepleted.

23. The method, the use, AKT inhibitor and/or an inhibitor of a PH domain protein for use or the population of immune cells of claim 22, wherein the subject has been lymphodepleted using lymphodepleting chemotherapy or radiation therapy.

24. The method of any one of claims 1 to 7 or 16 to 23, the use according to any one of claims 8 to 12 or 20 to 23, the AKT inhibitor and/or an inhibitor of a PH domain protein for use according to any one of claims 13 or 14 or 20 to 23 or the population of immune cells according to any one of claims 15 or 20 to 23, wherein the subject has a cancer, an infection, or an inflammatory disease.

25. The method, the use, AKT inhibitor and/or an inhibitor of a PH domain protein for use or the population of immune cells of claim 24, wherein the infection is a viral infection.

26. The method, the use, AKT inhibitor and/or an inhibitor of a PH domain protein for use or the population of immune cells of claim 24, wherein the subject has a cancer selected from colon cancer and breast cancer.

27. The method, the use, AKT inhibitor and/or an inhibitor of a PH domain protein for use or the population of immune cells of claim 24, wherein the subject has a cancer associated with low antigen abundance.

28. The method, the use, AKT inhibitor and/or an inhibitor of a PH domain protein for use or the population of immune cells of claim 27, wherein the subject has
i) acute myeloid leukaemia where low CD33+ blasts are dominant, or
ii) diffuse large B cell lymphoma or non-Hodgkin's lymphoma with low levels of CD19 and/or CD20.

29. The method of any one of claims 1 to 7 or 16 to 28, the use according to any one of claims 8 to 12 or 20 to 28, the AKT inhibitor and/or an inhibitor of a PH domain protein for use according to any one of claims 13 or 14 or 20 to 28 or the population of immune cells according to any one of claims 15 or 20 to 28, wherein the subject is a human.

30. An AKT inhibitor and/or an inhibitor of a PH domain protein for modifying a dendritic cell and/or natural killer cell response in a subject.

31. Use of a population of cells comprising dendritic cells and/or natural killer cells for modifying a dendritic cell and/or natural killer cell response in a subject, wherein the subject has been, or will be, administered with an AKT inhibitor and/or an inhibitor of a PH domain protein.

32. The method of any one of claims 1 to 7 or 16 to 29, the use according to any one of claim 8 to 12 or 20 to 29 or 31, the AKT inhibitor and/or an inhibitor of a PH domain protein for use according to any one of claims 13 or 14 or 20 to 30 or the population of immune cells according to any one of claims 15 or 20 to 29, wherein the AKT inhibitor and/or an inhibitor of a PH domain protein is selected from triciribine (TCN), triciribine 5′-monophosphate (TCN-P), AKT inhibitor VIII, MK-2206, AZD5363, GDC-0068, GSK2141795 and GSK2110183 hydrochloride.

33. The method of any one of claim 1 to 7 or 16 to 29 or 32, the use according to any one of claim 8 to 12 or 20 to 29 or 31, the AKT inhibitor and/or an inhibitor of a PH domain protein for use according to any one of claims 13 or 14 or 20 to 30 or the population of immune cells according to any one of claim 15 or 20 to 29 or 32, wherein the AKT inhibitor and/or an inhibitor of a PH domain protein inhibitor is triciribine (TCN) or triciribine 5′-monophosphate (TCN-P).

34. The method of any one of claims 1 to 7 or 16 to 29 or 32 to 33, the use according to any one of claims 8 to 12 or 20 to 29 or 31 to 33, the AKT inhibitor and/or an inhibitor of a PH domain protein for use according to any one of claims 13 or 14 or 20 to 30 or 32 to 33, or the population of immune cells according to any one of claims 15 or 20 to 29 or 32 to 33, wherein the dose of AKT inhibitor and/or an inhibitor of a PH domain protein administered to the subject is about 2 mg/kg.

35. The method, the use, the AKT inhibitor and/or an inhibitor of a PH domain protein for use or the population of immune cells according to claim 34, wherein the AKT inhibitor and/or an inhibitor of a PH domain protein is administered intravenously to the subject twice weekly.

36. The method, the use, the AKT inhibitor and/or an inhibitor of a PH domain protein for use or the population of immune cells according to claim 35, wherein the first dose of AKT inhibitor and/or an inhibitor of a PH domain protein is administered concurrently with administration of the immune cells and/or checkpoint blockade.

37. A method for modifying an immune response in a subject, preferably a T-cell response in a subject, the method comprising administering to the subject a checkpoint inhibitor, preferably an anti-PD-1 antibody, and an AKT inhibitor and/or an inhibitor of a PH domain protein, wherein the effect of the treatment is synergistic when compared to the individual effects of each treatment.

38. A method of producing a cell population comprising immune cells, the method comprising culturing immune cells in medium comprising an AKT inhibitor and/or an inhibitor of a PH domain protein, preferably wherein the immune cells are T-cells, dendritic cells, natural killer cells, macrophages, myeloid cells or a combination thereof.

39. The method of claim 38, wherein the immune cells are T-cells comprising a chimeric antigen receptor (CAR-T-cells).

40. The method of claim 39 which comprises
a) producing a T-cell enriched population of cells from a population of immune cells isolated from a subject;
b) transforming the T-cell enriched population of cells with a vector encoding a chimeric T-cell receptor; and
c) culturing the cells obtained in step b) in medium comprising an AKT inhibitor and/or an inhibitor of a PH domain protein.

41. The method of claim 39 or claim 40, wherein the CAR-T-cells produced using the method comprises central memory (T_{CM}) and/or stem cell (T_{SCM}) T-cells.

42. The method of claim 41, wherein the central memory (T_{CM}) and/or stem cell (T_{SCM}) T-cells comprise at least about 10% of the CAR-T-cells produced using the method.

43. The method of claim 42, wherein the central memory (T_{CM}) and/or stem cell (T_{SCM}) T-cells comprise between about 10% and about 30% of the CAR-T-cells produced using the method.

44. The method of any one of claims 41 to 43, wherein the central memory (T_{CM}) and/or stem cell (T_{SCM}) T-cells comprise at least about 0.8% of the CD8+ T-cells produced using the method.

45. The method of claim 44, wherein the central memory (T_{CM}) and/or stem cell (T_{SCM}) T-cells comprise between about 0.8% and about 5% of the CD8+ T-cells produced using the method.

46. The method of any one of claims 38 to 45, wherein the central memory (T_{CM}) and/or stem cell (T_{SCM}) T-cells comprise at least about 0.37% of the total lymphocytes produced using the method.

47. The method of claim 46, wherein the central memory (T_{CM}) and/or stem cell (T_{SCM}) T-cells comprise between about 0.37% and about 5% of the total lymphocytes produced using the method.

48. The method of any one of claims 41 to 47, wherein the T_{CM} cells include CD45RO+CD62L+ T-cells, preferably CD45RO+CD62L^{hi} T-cells.

49. The method of any one of claims 41 to 48, wherein the TS_{CM} cells include CD27⁺CD95⁺ T-cells.

50. The method of any one of claims 41 to 49, which further comprises enriching the cultured cells for the T_{CM} and/or T_{SCM} cells.

51. The method of any one of claims 38 to 50, wherein the method produces a greater percentage of central memory (T_{CM}) and/or stem cell (T_{SCM}) T-cells than T-cells cultured under identical conditions in the absence of the AKT inhibitor and/or an inhibitor of a PH domain protein.

52. The method of claim 51, wherein the method produces at least about 25% more central memory (T_{CM}) and/or stem cell (T_{SCM}) T-cells than T-cells cultured under identical conditions in the absence of the AKT inhibitor and/or an inhibitor of a PH domain protein.

53. The method of any one of claims 38 to 52, wherein the method produces a smaller percentage of regulatory (T_{REG}) T-cells than T-cells cultured under identical conditions in the absence of the AKT inhibitor and/or an inhibitor of a PH domain protein.

54. The method of claim 53, wherein the method produces at least about 5% less regulatory (T_{REG}) T-cells than T-cells cultured under identical conditions in the absence of the AKT inhibitor and/or an inhibitor of a PH domain protein.

55. The method of claim 53 or 54, wherein the T_{REG} cells are CD3+ CD4+ CD25+ FoxP3+ T-cells.

56. The method of any one of claims 39 to 55, wherein the chimeric antigen receptor binds a viral antigen.

57. The method of claim 56 which produces a population of CAR-T-cells with greater anti-viral activity than a population of CAR-T-cells cultured under identical conditions in the absence of the AKT inhibitor and/or an inhibitor of a PH domain protein.

58. The method of claim 38 which comprises
a) producing a dendritic cell enriched population of cells from a population of immune cells isolated from a subject;
b) exposing the cells from step a) to an antigen, and
c) culturing the cells in medium comprising an AKT inhibitor and/or an inhibitor of a PH domain protein.

59. The method of claim 38 or claim 58 which produces more dendritic cells than dendritic cells cultured under identical conditions in the absence of the AKT inhibitor and/or an inhibitor of a PH domain protein.

60. The method of claim 38 which comprises
a) producing a natural killer cell (NK) enriched population of cells from a population of immune cells isolated from a subject;
b) culturing the cells from step a) in medium comprising an AKT inhibitor and/or an inhibitor of a PH domain protein.

61. The method of claim 38 or claim 60 which produces a population of NK cells which have greater cytotoxic activity than a population of NK cells cultured under identical conditions in the absence of the AKT inhibitor and/or an inhibitor of a PH domain protein.

62. The method of any one of claims 38 to 61, wherein the cells are human cells.

63. The method of any one of claims 38 to 62, wherein the cultured cells, or a sub-population thereof comprising the immune cells, are administered to the subject.

64. A population of immune cells produced using a method according to any one of claims 40 to 63.

65. A cell population comprising CAR-T-cells, wherein at least 10% of the CAR-T-cells are CD8+ T_{CM} and/or T_{SCM} cells.

66. The cell population of claim 65 which has not been sorted.

67. The cell population of claim 65 or 66, wherein less than 25% of the CAR-T-cells are TREGS.

68. A pharmaceutical composition comprising the population of immune cells according to any one of claims 64 to 67.
